(19) United States
(12) Patent Application Publication
(10) Pub. No.: US 2025/0230482 A1
(43) Pub. Date: Jul. 17, 2025

(54) SYSTEM AND KIT FOR RAPID MULTIMODAL DETECTION OF ANAEROBIC SULFATE-REDUCING BACTERIA VIA INTEGRATIVE SPECTRAL COMPARISON

(71) Applicant: Field Water Testing, LLC, Bountiful, UT (US)

(72) Inventors: Christopher Lloyd, West Valley City, UT (US); Andrew Duncan, Logan, UT (US)

(21) Appl. No.: 19/014,749

(22) Filed: Jan. 9, 2025

Related U.S. Application Data
(60) Provisional application No. 63/620,128, filed on Jan. 11, 2024.

(51) Int. Cl. C12Q 1/26; C12M 1/34; C12M 3/00; C12Q 1/04; G01N 21/64

(52) U.S. Cl. CPC C12Q 1/26; C12M 23/42; C12M 41/30; C12Q 1/04; G01N 21/6428; G01N 2021/6439; G01N 2333/90212

(57) ABSTRACT

A system for rapid multimodal detection of anaerobic SRB in a fluid test sample via integrative spectral comparison includes a compact, mini, or advanced assay reader with sample compartments configured to receive assay samples for comparison with respective spectral reference standards. The assay reader includes one or more excitation sources configured to emit light at wavelengths selected to facilitate colorigenic and/or fluorogenic analysis of the assay samples. The system includes a kit with one or more first reagents formulated to form a first assay sample indicating a level of H₂S in the fluid test sample, and one or more second reagents formulated to form a second assay sample indicating a level of enzymatic activity of anaerobic SRB species. The kit also includes one or more oxygen scavengers to maintain an anaerobic environment.

### CROSS-REFERENCE TO RELATED APPLICATIONS

This application claims priority to U.S. Provisional Patent Application No. 63/620,128 titled “System and Method for Detecting Sulfate-reducing Bacteria in a Fluid” filed Jan. 11, 2024, which is hereby incorporated by reference to the extent legally allowable.

### FIELD

The subject matter disclosed herein relates to detecting sulfate-reducing bacteria in a fluid and more particularly relates to a system, assay kit, and method for field use for rapid multimodal detection of anaerobic sulfate-reducing bacteria via integrative spectral comparison.

### BACKGROUND

Sulfate-reducing bacteria (SRB) are prokaryotic microorganisms which can utilize sulfate (So₄²⁻) in anaerobic respiration and produces hydrogen sulfide (H₂S). H₂S produced by SRB can be corrosive, malodorous, and/or toxic and/or can form metal sulfide scales that damage equipment, pipelines, and wells. As such, detection of SRB can help to prevent such consequences.

### SUMMARY

In some aspects, the techniques described herein relate to a system for field use in rapid multimodal detection of anaerobic sulfate-reducing bacteria (SRB) in a fluid test sample via integrative spectral comparison, the system including: an assay reader including: one or more sample compartments configured to receive: one or more assay samples for comparison with respective spectral reference standards; and one or more excitation sources configured to emit light at wavelengths selected to facilitate colorigenic and/or fluorogenic analysis of one or more assay samples; and a kit for performing an integrative assay of anaerobic SRB-mediated components of the fluid test sample including: one or more first reagents, the one or more first reagents formulated to form a first assay sample upon contact with a first portion of the fluid test sample and to indicate, via a first set of spectral parameters, a level of hydrogen sulfide (H₂S) in the fluid test sample; one or more second reagents, the one or more second reagents formulated to form a second assay sample upon mixing with a second portion of the fluid test sample and to indicate, via a second set of spectral parameters, a level of enzymatic activity of one or more species of anaerobic SRB in the fluid test sample; and one or more oxygen scavengers combined with the one or more second reagents and formulated to, upon contact with the fluid test sample, maintain an anaerobic environment for the one or more species of anaerobic SRB.

In various aspects, the techniques described herein relate to a system, wherein the assay reader includes one or more windows through which one or more spectral parameters of the first set of spectral parameters and/or the second set of spectral parameters pertaining to the first assay sample and/or the second assay sample are determinable.

In certain aspects, the techniques described herein relate to a system, wherein: the one or more first reagents are formulated to react with H₂S to yield a first fluorescent compound; and the one or more second reagents are formulated to yield a second fluorescent compound when exposed to one or more species of anaerobic sulfate-reducing bacteria in the fluid test sample.

In one or more aspects, the techniques described herein relate to a system, wherein the first set of spectral parameters and the second set of spectral parameters include visible fluorescence emitted from the fluid test sample by the first fluorescent compound and the second fluorescent compound in response to excitation by an ultraviolet (UV) light source.

In some aspects, the techniques described herein relate to a system, wherein an integrative assay sample is formed upon contact with the fluid test sample into an individual assay container which contains the one or more first reagents for detecting H₂S and the one or more second reagents for detecting enzymatic activity of SRB.

In various aspects, the techniques described herein relate to a system, wherein the assay reader includes a spectrometry instrument configured to detect and analyze the first set of spectral parameters and the second set of spectral parameters pertaining to the integrative assay sample concurrently.

In certain aspects, the techniques described herein relate to a system, wherein at least one of the one or more windows includes a filter that selectively filters light corresponding to excitation source wavelengths and passes light corresponding to excitation response wavelengths.

In one or more aspects, the techniques described herein relate to a system, wherein the filter selectively filters UV excitation and passes visible light corresponding to excitation response wavelengths.

In some aspects, the techniques described herein relate to a kit, including: one or more first reagents formulated to indicate via a first set of spectral parameters a level of hydrogen sulfide (H₂S) in a fluid test sample; one or more second reagents formulated to indicate via a second set of spectral parameters a level of enzymatic activity of one or more species of anaerobic sulfate-reducing bacteria (SRB) in the fluid test sample; and one or more oxygen scavengers combined with the one or more second reagents and formulated to, upon contact with the fluid test sample, provide an anaerobic environment for the one or more species of anaerobic sulfate-reducing bacteria.

In various aspects, the techniques described herein relate to a kit, wherein one or more of the one or more first reagents are formulated to produce the first set of spectral parameters having a longer wavelength range than an excitation source wavelength range used in connection with the one or more second reagents.

In certain aspects, the techniques described herein relate to a kit, wherein at least one spectral parameter of the first set of spectral parameters and the second set of spectral parameters includes a wavelength of light emitted in response to excitation by an ultraviolet (UV) light source.

In one or more aspects, the techniques described herein relate to a kit, wherein the one or more oxygen scavengers include sodium dithionite.

In some aspects, the techniques described herein relate to a kit, further including (2S)-2-amino-5-[[(2R)-1-(carboxymethylamino)-1-oxo-3-sulfanylpropan-2-yl]amino]-5-oxopentanoic acid that in combination with sodium dithionite synergistically enhances oxygen-scavenging properties of the combination.

In various aspects, the techniques described herein relate to a kit, wherein the one or more second reagents include (6′-acetyloxy-3-oxospiro[2-benzofuran-1,9′-xanthene]-3′-yl) acetate, and the first set of spectral parameters includes emitted visible light in response to excitation by a UV light source.

In certain aspects, the techniques described herein relate to a kit, wherein the one or more second reagents are formulated to indicate the level of enzymatic activity of one or more species of anaerobic sulfate-reducing bacteria in the fluid test sample and are selected from a group consisting of: 3,6-diacetoxyfluoran, 7-Hydroxy-3-oxo-3H-phenoxazine-10-oxide, dipeptidyl-7-Amino-4-methylcoumarin, p-Nitrophenyl phosphate, and/or 5-Bromo-6-chloro-3-indolyl caprylate.

In one or more aspects, the techniques described herein relate to a kit, wherein the one or more first reagents are formulated to indicate levels of H₂S in the fluid test sample by the first set of spectral parameters with a predetermined spectral wavelength range upon reaction with H₂S in the fluid test sample.

In some aspects, the techniques described herein relate to a kit, wherein the one or more first reagents are formulated to include alpha-naphthol orange for enhancing a spectral comparison of a reaction of H₂S with the alpha-naphthol orange, where the fluid test sample before contacting the one or more first reagents produced a yellow baseline color and/or a target wavelength to be detected is in a range of about 550 nanometers (nm) to about 590 nm.

In various aspects, the techniques described herein relate to a kit, wherein the one or more first reagents are formulated to include tetrasodium and 4-amino-6-[[4-[4-[(8-amino-1-hydroxy-5,7-disulfonatonaphthalen-2-yl)diazenyl]-3-methylphenyl]-2-methylphenyl]diazenyl]-5-hydroxynaphthalene-1,3-disulfonate for enhancing detection of concentrated H₂S.

In certain aspects, the techniques described herein relate to a kit, wherein the one or more first reagents include 5-[(3-carboxy-4-nitrophenyl)disulfanyl]-2-nitrobenzoic acid and the kit further includes one or more buffering agents 160.

In one or more aspects, the techniques described herein relate to a kit, wherein the one or more first reagents include 7-azido-4-methylchromen-2-one.

In some aspects, the techniques described herein relate to a kit, wherein the one or more first reagents and/or the one or more second reagents are lyophilized until contact with the fluid test sample.

In various aspects, the techniques described herein relate to a kit, further including a spectral reference standard, the spectral reference standard provided to indicate, via at least one of the first set of spectral parameters and the second set of spectral parameters corresponding respectively to wavelengths indicative of reaction of the one or more first reagents with at least one of a baseline level of H₂S and to wavelengths indicative of reaction of the one or more second reagents with a baseline level of enzymatic activity of the one or more species of anaerobic sulfate-reducing bacteria.

In certain aspects, the techniques described herein relate to a method for providing a kit for field use in rapid multimodal detection of anaerobic sulfate-reducing bacteria (SRB) in a fluid test sample via integrative spectral comparison, the method including: determining a first set of spectral parameters for one or more first reagents formulated to indicate, via the first set of spectral parameters, one or more predetermined levels of hydrogen sulfide (H₂S) in a fluid test sample; determining a second set of spectral parameters for one or more second reagents formulated to indicate, via the second set of spectral parameters, one or more predetermined levels of enzymatic activity of one or more species of anaerobic sulfate-reducing bacteria in the fluid test sample; providing a first spectral reference standard including a set of spectral parameters representative of one or more predetermined levels of hydrogen sulfide (H₂S) in a first spectral reference configured for performing a first comparison with a first spectral signal to be generated in response to a reaction of the one or more first reagents with H₂S upon contact with the fluid test sample; and providing a second spectral reference standard including a set of spectral parameters representative of or more predetermined levels of enzymatic activity of one or more species of anaerobic sulfate-reducing bacteria in a second spectral reference based on a second comparison with a second spectral signal generated in response to microbial interaction with a combination of the one or more second reagents and one or more oxygen scavengers that are formulated to, upon contact with the fluid test sample, maintain an anaerobic environment for the one or more species of anaerobic sulfate-reducing organisms.

In one or more aspects, the techniques described herein relate to a method, further including indicating one or more mitigation recommendations to reduce levels of anaerobic sulfate-reducing bacteria in a source of fluid for the fluid test sample based on a combination of information determined from the first comparison and the second comparison.

### BRIEF DESCRIPTION OF THE DRAWINGS

A more particular description of the techniques briefly described above will be rendered by reference to specific embodiments that are illustrated in the appended drawings. Understanding that these drawings depict only some embodiments and are not, therefore, to be considered to be limiting of scope, the embodiments will be described and explained with additional specificity and detail through the use of the accompanying drawings, in which:

FIG. 1 is a schematic block diagram illustrating a system for field use in rapid multimodal detection of anaerobic sulfate-reducing bacteria (SRB) in a fluid test sample via integrative spectral comparison, according to one or more aspects of the present disclosure;

FIG. 2 (Prior Art) is an overview diagram 200 illustrating various approaches for assessing indications of SRB in fluid test samples, according to one or more aspects of the present disclosure;

FIG. 3 is a schematic block diagram illustrating a system for field use in rapid multimodal detection of anaerobic SRB in a fluid test sample via integrative spectral comparison of two or more assay samples prepared with first reagents formulated to detect H₂S and second reagents with oxygen scavengers formulated to detect anaerobic SRB and maintain an anaerobic environment, according to one or more aspects of the present disclosure;

FIG. 4 is a schematic block diagram illustrating a system for field use in rapid multimodal detection of anaerobic SRB in a fluid test sample via integrative spectral comparison of spectral parameters resulting from a combination assay sample, according to one or more aspects of the present disclosure;

FIG. 5 is a graph illustrating working Example 1 results of H₂S-related spectral response parameters using a reagent designated T1-Rgt2 in Table 1 of the specification for integrative spectral comparison with selected anerobic SRB-spectral response parameters, according to one or more aspects of the present disclosure;

FIG. 6 is a graph illustrating working Example 2 results of H₂S-related spectral response parameters using a reagent designated T2-Rgt1 in Table 2 of the specification for integrative spectral comparison with selected anerobic SRB-spectral response parameters, according to one or more aspects of the present disclosure;

FIG. 7 is a graph illustrating working Example 3 results of H₂S-related spectral response parameters using a reagent designated T1-Rgt1 in Table 1 of the specification for integrative spectral comparison with selected anerobic SRB-spectral response parameters, according to one or more aspects of the present disclosure;

FIG. 8 is a graph illustrating working Example 4 results of H₂S-related spectral response parameters using a reagent designated T1-Rgt3 in Table 1 of the specification for integrative spectral comparison with selected anerobic SRB-spectral response parameters, according to one or more aspects of the present disclosure;

FIG. 9 is a graph illustrating working Example 5 of an anaerobic SRB-related spectral response parameters using T3-Rgt1 for integrative spectral comparison with a selected H₂S-related spectral response parameters, according to one or more aspects of the present disclosure;

FIG. 10 is a graph illustrating working Example 6 of H₂S-related spectral response parameters that stimulate anaerobic SRB-related spectral response parameters using an integrative combination assay T1-Rgt3 for integrative spectral comparison with an anaerobic SRB-related spectral response parameters, according to one or more aspects of the present disclosure;

FIG. 11 is a schematic flowchart diagram illustrating a method for detecting H₂S, according to one or more aspects of the present disclosure;

FIG. 12 is a schematic flowchart diagram illustrating a method for detecting H₂S via one or more lyophilized reagents, according to one or more aspects of the present disclosure;

FIG. 13 is a schematic flowchart diagram illustrating a method for detecting H₂S via one or more assay containers, according to one or more aspects of the present disclosure;

FIG. 14 is a schematic flowchart diagram illustrating a method for detecting H₂S via two or more assay containers, according to one or more aspects of the present disclosure; and

FIG. 15 is a schematic flowchart diagram illustrating a method for detecting H₂S via one or more reagents selected via a predicted H₂S level, according to one or more aspects of the present disclosure.

### DETAILED DESCRIPTION

### Disclosure Reading Guidelines

As will be appreciated by one skilled in the art, aspects of the disclosure can be implemented as a system, method or program product. Accordingly, aspects or implementations can take the form of an entirely hardware implementation, an entirely software implementation (including firmware, resident software, micro-code, etc.) or an implementation combining software and hardware aspects that can all generally be referred to herein as a “circuit,” “module,” “controller,” or “system.” Furthermore, aspects of the disclosed subject matter can take the form of a program product implemented in one or more computer readable storage devices storing machine-readable code, computer readable code, and/or program code, referred hereafter as code.

For the purposes of this disclosure, the term “computer readable storage devices” refers to non-transitory, tangible, physical computer hardware configured to store computer-executable instructions or data. This specifically includes, but is not limited to, hardware such as hard disk drives, solid-state drives, flash memory devices, optical discs, magnetic tapes, and other similar physical storage media. Furthermore, the term “Computer readable storage devices” expressly excludes transitory propagating signals per se, carrier waves, electromagnetic transmissions, and any other form of signal transmission, regardless of whether such signals embody or encode computer-executable instructions or data. This definition is intended to distinguish clearly between physical storage hardware (computer readable storage devices) and the transmission of signals (computer readable transmission media), ensuring compliance with Ex parte Mewherter, 107 USPQ2d 1857 (PTAB 2013) (precedential) and relevant U.S. Patent & Trademark Office Guidance for Evaluating Subject Matter Eligibility Under 35 USC § 101.

To the extent that computer readable storage devices are disclosed and claimed, such disclosure relates solely to the configuration and operation of tangible, physical storage hardware and not to abstract or transitory signal transmission.

Certain of the functional units described in this specification have been labeled as modules or controllers, in order to more particularly emphasize the implementation options that can be used. For example, some functions of a module or a controller can be implemented as a hardware circuit comprising custom VLSI circuits or gate arrays, off-the-shelf semiconductors such as logic chips, transistors, or other discrete components. A module or controller can also be implemented in programmable hardware devices such as field-programmable gate arrays, programmable array logic, programmable logic devices or the like.

Various modules or controllers can also be implemented, partially or wholly, in code and/or software for execution by various types of processors. An identified controller or module of code can, for instance, comprise one or more physical or logical blocks of executable code which can, for instance, be organized as an object, procedure, or function. In one or more examples, the executables of an identified controller or module need not be physically located together but can comprise disparate instructions stored in different locations which, when joined logically together, comprise the module and achieve the stated purpose for the controller or module.

Indeed, a controller or a module of code can be a single instruction, or many instructions, and can even be distributed over several different code segments, among different programs, and across several memory devices. Similarly, operational data can be identified and illustrated herein within modules and can be embodied in any suitable form and organized within any suitable type of data structure. The operational data can be collected as a single data set or can be distributed over different locations including over different computer readable storage devices. Where a controller, module or portions thereof are implemented in software, the software portions are stored on one or more computer readable storage devices.

Any combination of one or more computer readable medium can be utilized. The computer readable medium can be a computer readable storage medium. The computer readable storage medium can be a storage device storing the code. The storage device can be, for example, but not limited to, an electronic, magnetic, optical, electromagnetic, infrared, holographic, micromechanical, or semiconductor system, measurement apparatus, or device, or any suitable combination of the foregoing.

More specific examples (a non-exhaustive list) of the storage device would include the following: an electrical connection having one or more wires, a portable computer diskette, a hard disk, a random access memory (RAM), a read-only memory (ROM), an erasable programmable read-only memory (EPROM or Flash memory), a portable compact disc read-only memory (CD-ROM), an optical storage device, a magnetic storage device, or any suitable combination of the foregoing. In the context of this document, a computer readable storage medium can be any tangible medium that can contain or store a program for use by or in connection with an instruction execution system, measurement apparatus, or device.

Code for carrying out operations for some implementations can be written in any combination of one or more programming languages including an object-oriented programming language such as Python, Ruby, Java, Smalltalk, C++, or the like, and conventional procedural programming languages, such as the “C” programming language, or the like, and/or machine languages such as assembly languages. The code can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider).

Reference throughout this specification to “one aspect,” “an aspect,” or similar language means that a particular feature, structure, or characteristic described in connection with the aspect is included in at least one implementation. Thus, appearances of the phrases “in one implementation,” “in an implementation,” and similar language throughout this specification can, but do not necessarily, all refer to the same implementation, but mean “one or more but not all implementations” unless expressly specified otherwise. The terms “including,” “comprising,” “having,” and variations thereof mean “including but not limited to,” unless expressly specified otherwise. An enumerated listing of items does not imply that any or all of the items are mutually exclusive unless expressly specified otherwise. The terms “a,” “an,” and “the” also refer to “one or more” unless expressly specified otherwise.

Furthermore, the described features, structures, or characteristics of the aspects or implementations can be combined in any suitable manner. In the following description, numerous specific details are provided, such as examples of programming, software modules, user selections, network transactions, database queries, database structures, hardware modules, hardware circuits, hardware chips, etc., to provide a thorough understanding of aspects and implementations. One skilled in the relevant art will recognize, however, that an implementation can be practiced without one or more of the specific details, or with other methods, components, materials, and so forth. In other instances, well-known structures, materials, or operations are not shown or described in detail to avoid obscuring aspects of the implementation.

Aspects of the disclosed implementations are described below with reference to schematic flowchart diagrams and/or schematic block diagrams of methods, measurement apparatuses, systems, and program products according to examples. It will be understood that some blocks of the schematic flowchart diagrams and/or schematic block diagrams, and combinations of blocks in the schematic flowchart diagrams and/or schematic block diagrams, can be implemented by code. This code can be provided to a processor of a general-purpose computer, special purpose computer, or other programmable data processing measurement apparatus to produce a machine, such that the instructions, which execute via the processor of the computer or other programmable data processing measurement apparatus, create means for implementing the functions/acts specified in the schematic flowchart diagrams and/or schematic block diagrams block or blocks.

The schematic flowchart diagrams and/or schematic block diagrams in the Figures illustrate the architecture, functionality, and operation of possible implementations of measurement apparatuses, systems, methods and program products according to various embodiments. In this regard, each block in the schematic flowchart diagrams and/or schematic block diagrams can represent a module, segment, or portion of code, which comprises one or more executable instructions of the code for implementing the specified logical function(s).

It should also be noted that, in some alternative implementations, the functions noted in the block can occur out of the order noted in the Figures. For example, two blocks shown in succession can, in fact, be executed substantially concurrently, or the blocks can sometimes be executed in the reverse order, depending upon the functionality involved. Other steps and methods can be conceived that are equivalent in function, logic, or effect to one or more blocks, or portions thereof, of the illustrated Figures.

Although various arrow types and line types can be employed in the flowchart and/or block diagrams, they are understood not to limit the scope of the corresponding aspects or implementations. Indeed, some arrows or other connectors can be used to indicate only the logical flow of the depicted example aspect. For instance, an arrow can indicate a waiting or monitoring period of unspecified duration between enumerated steps of the depicted example implementation. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and code.

The description of elements in each FIG. can refer to elements of proceeding figures. Unless expressly noted or otherwise clear from context, like numbers refer to like elements in all figures, including alternate implementation involving like elements.

As used herein, a list using the conjunction of “and/or” includes any single item in the list or a combination of items in the list. For example, a list of A's, B and/or C includes only A, only B, only C, a combination of A and B, a combination of B and C, a combination of A and C or a combination of A, B and C. As used herein, a list using the terminology “one or more of” includes any single item in the list or a combination of items in the list. For example, one or more of A, B and C includes only A, only B, only C, a combination of A and B, a combination of B and C, a combination of A and C or a combination of A, B and C. As used herein, a list using the terminology “one of” includes one and only one of any single items in the list. For example, “one of A, B and C” includes only A, only B or only C and excludes combinations of A, B and C. As used herein, “a member selected from the group consisting of A, B, and C,” includes one and only one of A, B, or C, and excludes combinations of A, B, and C. As used herein, “a member selected from the group consisting of A, B, and C and combinations thereof” includes only A, only B, only C, a combination of A and B, a combination of B and C, a combination of A and C or a combination of A, B, and C.

Unless otherwise defined, all technical and scientific terms used herein have the same meaning as commonly understood by one of ordinary skill in the art to which the subject matter of this disclosure belongs. The terminology used in the detailed description herein is for describing particular example implementations only and, unless otherwise clear from context, is not intended to be limiting of the scope of the subject matter. As used in the description and the appended claims, the singular forms “a,” “an,” and “the” are intended to include the plural forms as well, unless the context clearly indicates otherwise.

The term “about” as used in this disclosure can allow for a degree of variability in a value or range, for example, within 10%, within 5%, or within 1% of a stated value or of a stated limit of a range.

All numbers expressing quantities of ingredients, reaction conditions, detection wavelength ranges and so forth used in the specification and claims are to be understood as being modified expressly or implicitly in all instances by the term “about.” Accordingly, unless indicated to the contrary, the numerical parameters set forth in the specification and attached claims are approximations that can vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosed subject matter are approximations, the numerical values set forth in the specific examples are reported reasonably precisely. Every numerical range given throughout this specification will include every narrower numerical range that falls within such broader numerical range, as if such narrower numerical ranges were all expressly written herein.

The term “substantially” as used in this disclosure refers to a majority of, or mostly, as in at least about 50%, 60%, 70%, 80%, 90%, 95%, 96%, 97%, 98%, 99%, 99.5%, 99.9%, 99.99%, or at least about 99.999% or more.

### Overview and Introduction

Current methods for detecting H₂S in a fluid test sample rely on the detection of H₂S in that sample, typically via the production of insoluble sulfide salts. This method suffers from interference from H₂S that has arisen from inorganic origins or from past SRB activity. In some examples, SRB in the sample are currently not viable, so the detection of H₂S is not as meaningful in such examples as in examples in which the SRB are currently viable. Additionally, current methods of detecting SRB have long incubation periods. Examples of the present disclosure include methods for detecting H₂S that can help to improve testing speed and/or more accurately tie the detection of H₂S to active SRB in the fluid test sample.

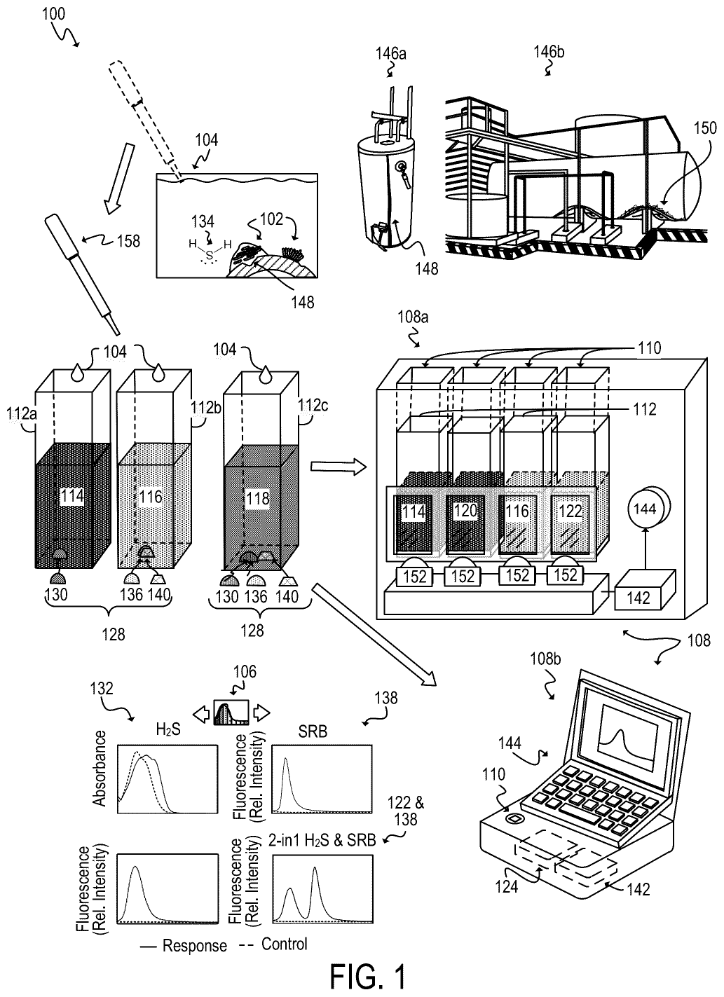

FIG. 1 is a schematic block diagram illustrating portable system and kit for rapid multimodal detection of sulfate-reducing bacteria via integrative spectral comparison, according to one or more aspects of the present disclosure.

The present disclosure relates to a portable field system designed for the rapid and multimodal detection of anaerobic sulfate-reducing bacteria (SRB) in a fluid test sample using integrative spectral comparison. The system achieves enhanced specificity and accuracy by correlating distinct spectral parameters associated with both hydrogen sulfide (H₂S) detection and SRB enzymatic activity. This integration allows differentiation between biologically mediated H₂S production and non-biological or abiotic sources of H₂S, overcoming limitations in conventional SRB detection methods.

The term “integrative” as used herein refers to an approach that combines data, measurements, or analytical outputs from distinct sources or methods into a unified framework to enhance specificity, accuracy, or interpretive value. In the context of the disclosed invention, integrative methods or systems synthesize results from multiple, complementary assays to identify or quantify specific target properties with greater precision than individual methods used independently. For example, in the disclosed system, spectral parameters derived from the detection of hydrogen sulfide (H₂S) are combined with spectral parameters obtained from enzymatic activity assays specific to sulfate-reducing bacteria (SRB). This integration provides a more specific and accurate indication of SRB activity by correlating signals from biologically derived H₂S production and SRB enzymatic activity. Non-integrative approaches, by contrast, rely on a single data source or uncorrelated outputs, such as measuring H₂S levels without considering the biological origin or performing independent enzymatic activity assays without correlation to H₂S production.

“Integrative spectral comparison” refers to a specific implementation of integrative methods where spectral parameters from distinct assays are compared, correlated, or otherwise analyzed to distinguish between biologically mediated and abiotic phenomena. For instance, in the disclosed invention, a colorigenic reagent detecting H₂S (e.g., alpha-naphthol orange) generates a spectral response corresponding to a peak absorbance wavelength of ˜580 nm. Simultaneously, a fluorogenic reagent detecting SRB enzymatic activity (e.g., (6′-acetyloxy-3-oxospiro[2-benzofuran-1,9′-xanthene]-3′-yl) acetate) generates a spectral response with a fluorescence peak at ˜530 nm. By comparing these spectral outputs within the assay reader, the system determines whether the detected H₂S is biologically derived from active SRB or originates from non-biological sources. This integration enables greater specificity by correlating enzymatic activity with H₂S production. A non-integrative spectral approach, such as measuring a single spectral parameter or separately analyzing enzymatic activity and H₂S levels without comparing the outputs, would fail to achieve the enhanced specificity provided by the disclosed invention.

The term “integrative combination assay” describes the methodology of performing multiple assays in a coordinated manner to simultaneously detect distinct but related properties of a sample and then combining these results for a unified interpretation. The disclosed technologies can involve the use of a combined assay that can be combined at the assay reagent level and/or at the spectral response level with first reagents formulated to react with H₂S, generating a detectable spectral change, and second reagents combined with oxygen scavengers to maintain an anaerobic environment and detect SRB enzymatic activity. The assay components can be performed concurrently or sequentially in the same assay sample, and the spectral response outputs are integrated to determine whether the H₂S detected is biologically mediated by viable anaerobic SRB

An approach where the two assays are performed independently without combining H₂S and SRB reagents or comparing their spectral outputs would not constitute an integrative combination assay. Similarly, a method that relies solely on detecting H₂S without distinguishing its biological origin, or that only measures biomarkers associated with anaerobic SRB without taking steps to help maintain an anaerobic environment or without assessing and indication of viability such as metabolic activity indicated by levels of enzymatic activity to assessing the SRB component spectral response to the H₂S component spectral response, would not meet the definition.

The integrative technologies disclosed herein provide correlation and synthesis of multiple assay components and spectral responses to enhance specificity and interpretive accuracy. Examples from the specification demonstrate how integrative spectral comparison and combination assays achieve this by leveraging distinct but complementary measurements of H₂S and anaerobic SRB enzymatic/metabolic activity. Non-integrative methods lack this correlation and fail to provide the specificity and accuracy achieved by the disclosed system.

The system includes an assay reader configured for field use, which incorporates one or more sample compartments designed to securely hold assay samples during analysis. These sample compartments align with respective spectral reference standards, enabling direct comparison of spectral outputs. The assay reader further comprises one or more excitation sources 152 that emit light at wavelengths optimized for colorigenic and fluorogenic assays. For instance, UV light sources are used to excite fluorogenic reagents for detecting enzymatic activity, while visible light sources facilitate colorimetric analysis of H₂S levels. The reader may also include integrated filters to isolate specific wavelengths corresponding to reagent emission or absorbance peaks, ensuring accurate and reliable spectral measurements.

Among the advantageous and innovative aspects of the system is the integrative assay kit, which contains reagents used to perform the analysis. The kit includes first reagents formulated to detect H₂S within the fluid test sample. Upon contact with a portion of the fluid test sample, these reagents produce a measurable signal-such as a colorimetric or fluorogenic response-indicative of H₂S concentration. Non-limited examples of suitable first reagents include alpha-naphthol orange, which undergoes a visible color shift in the presence of H₂S, or 7-azido-4-methylchromen-2-one, which fluoresces upon reacting with H₂S. Further examples are provided in the detailed description below. Notably, in various examples, the one or more first reagent, the one or more second reagents, and/or the one more oxygen scavengers and corresponding enhancing agents are formulated and processed to be stable and optimized for use in variable environmental conditions encountered during field testing.

In certain examples, kit further includes second reagents designed to detect enzymatic activity indicative of metabolic active of viable anaerobic SRB. Such activity can include, for example, activity of esterases, oxidoreductases, and/or peptidases. Upon mixing with another portion of the fluid test sample, these reagents produce a distinct measurable signal associated with enzymatic activity. For example, (6′-acetyloxy-3-oxospiro[2-benzofuran-1,9′-xanthene]-3′-yl) acetate fluoresces when cleaved by SRB esterases, while 7-hydroxy-10-oxidophenoxazin-10-ium-3-one changes color in response to redox activity. The second reagents are combined with one or more oxygen scavengers to maintain an anaerobic environment essential for SRB viability and enzymatic function. Suitable oxygen scavengers include sodium dithionite, which chemically removes dissolved oxygen, and reduced glutathione, which synergistically enhances the oxygen-scavenging properties of sodium dithionite.

In some examples, to perform the assay, the fluid test sample is divided into two portions. The first portion is combined with the first reagents to form a first assay sample that detects H₂S, while the second portion is mixed with the second reagents and oxygen scavengers to form a second assay sample that measures SRB enzymatic activity. Both assay samples are then placed into the assay reader's sample compartments. In certain examples, a single aliquot of the fluid test sample is mixed with the first reagents, the second reagents, and the oxygen scavenger in a single assay container which is then placed into the assay reader's sample compartment for comparison with one or more tangible spectral reference standards and/or with one or more digital spectral reference standards such as can be utilized with a spectrometry instrument.

The excitation sources within the reader activate the reagents, producing measurable spectral responses. The first assay sample generates spectral parameters corresponding to H₂S levels, such as an absorbance peak at ˜580 nm or fluorescence at ˜450 nm. The second assay sample produces spectral parameters indicative of enzymatic activity, such as fluorescence at ˜530 nm.

The assay reader performs an integrative spectral comparison by analyzing the spectral responses of the H₂S assay components and anaerobic SRB assay components in conjunction with respective spectral reference standards. This comparison enhances the specificity of SRB detection by correlating the presence of H₂S with SRB enzymatic activity. For example, a detected H₂S signal accompanied by enzymatic activity confirms biologically mediated H₂S production by active anaerobic SRB, while the absence of enzymatic activity can suggest an abiotic source of H₂S. Conversely, enzymatic activity without significant H₂S detection may indicate that bacteria in the test sample are metabolically active but not currently producing detectable levels of H₂S.

This integrative approach provides a rapid and specific analysis of SRB activity in the fluid test sample, with results displayed visually, digitally, or in a report format suitable for field decision-making. The portable design of the system allows its deployment in various industrial, residential, and environmental settings, including oilfield pipelines, water treatment facilities, and residential plumbing systems. The system's ability to integrate spectral data ensures accurate differentiation between biologically and non-biologically mediated H₂S production, significantly advancing the field of microbial diagnostics and environmental monitoring.

FIG. 1 is a schematic block diagram illustrating a system 100 for field use in rapid multimodal detection of anaerobic sulfate-reducing bacteria (SRB) in a fluid test sample via integrative spectral comparison, according to one or more aspects of the present disclosure.

As discussed in the introduction, the inventors of the subject matter disclosed herein have developed a system 100 that is portable for field use for rapid multimodal detection of anaerobic SRB 102 in a fluid test sample 104 via integrative spectral comparison 106.

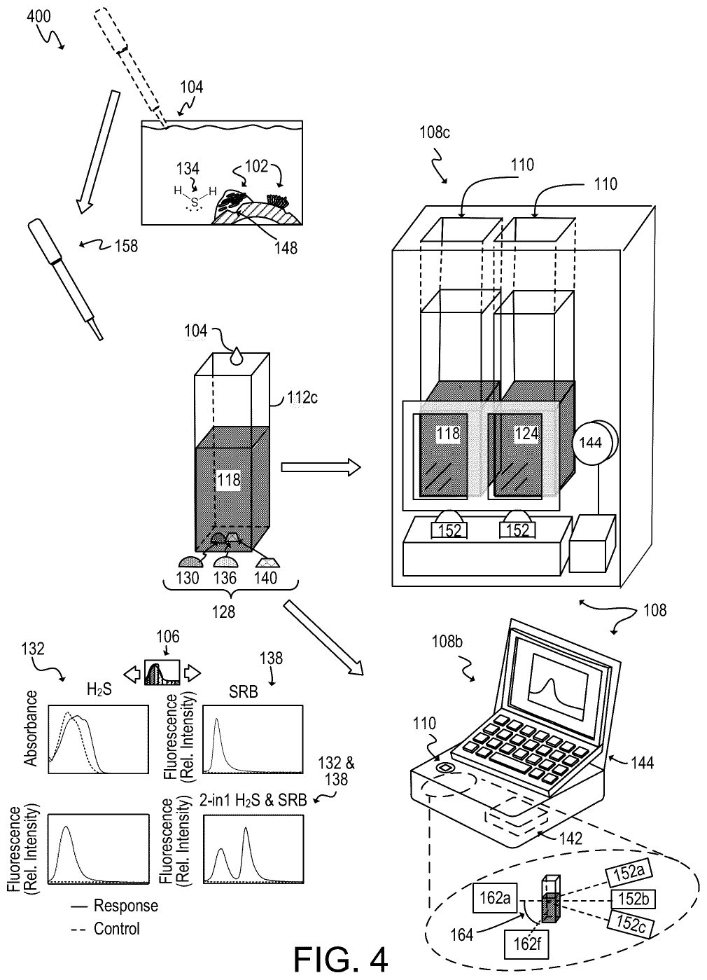

In various implementations, the system 100 includes an assay reader 108 with one or more sample compartments 110 configured to receive one or more assay containers 112 containing a first assay sample 114, a second assay sample 116, or a combination assay sample 118 for integrative spectral comparison 106 respectively with a first spectral reference standard 120, a second spectral reference standard 122, or a combined spectral reference standard 124, which in as depicted in this FIG. 1, may be a digital spectral reference standard 124 storage in a computer readable storage device accessed by the controller 142 of the advanced spectrometry assay reader 108b or the combined spectral reference standard can be a tangible spectral reference standard such as the combined spectral reference standard 124 described and depicted below with respect to FIG. 4.

In some implementations, the assay reader 108 includes one or more excitation sources 152 configured to emit light in a wavelength range selected to facilitate colorigenic analysis and/or fluorogenic analysis of one or more assay samples 114, 116, 118.

In some implementations, the assay reader 108 includes a controller 142 and a user interface 144.

In certain implementations, the assay reader 108 is a compact assay reader 108a with one to ‘2n’ sample compartments configured to into which one to ‘n’ assay samples 114, 116, 118 and one to ‘n’ tangible spectral reference standards 120, 122, 124 are insertable.

Notably, with an integrative combination assay, such a compact assay reader 108a can be used in the field to rapidly perform an integrative spectral comparison of both H₂S and anaerobic SRB enzymatic activity responses concurrently using a single combination assay sample 118 and a single spectral reference standard 124.

Further details about integrative spectral comparison of both H₂S and anaerobic SRB enzymatic activity responses concurrently using a single combination assay sample 118 are provided below with respect to the description of FIG. 4.

In various implementations, the system 100 includes a kit 128 for performing an integrative assay of anaerobic SRB-mediated components of the fluid test sample 104. For example, viable anaerobic SRB cam obtain energy by reducing sulfate (SO₄²⁻) to hydrogen sulfide (H₂S) in the absence of oxygen, thus H₂S thus reduced can be considered an SRB-mediated components of the fluid test sample 104. Similarly, certain enzymatic activity from enzymes such as esterases, oxidoreductases, and/or peptidases can indicate metabolic activity of viable anaerobic SRB.

In some implementations, the kit 128 includes one or more first reagents 130, where the one or more first reagents 130 are formulated to form a first assay sample 114 upon contact with a first portion of the fluid test sample 104 and to indicate, via a first set of spectral parameters 132, a level of H₂S 134 in the fluid test sample 104.

In certain implementations, the kit 128 include one or more second reagents 136, where the one or more second reagents 136 are formulated to form a second assay sample 116 upon mixing with a second portion of the fluid test sample 104 and to indicate, via a second set of spectral parameters 138, a level of enzymatic activity of one or more species of anaerobic SRB 102 in the fluid test sample 104; and one or more oxygen scavengers 140 combined with the one or more second reagents 136 and formulated to, upon contact with the fluid test sample (104), maintain an anaerobic environment for the one or more species of anaerobic SRB 102.

The inventors of the present disclosure have developed the system 100 and kit 128 as innovative technologies for multimodal detection of anaerobic sulfate-reducing bacteria via integrative spectral comparison so that various problems associated with SRB and its byproducts can be more readily detected.

In certain implementations, methods of producing the system, and or kit include providing the first and second reagents and the oxygen scavengers, along with instructions for performing one or more integrative spectral comparisons of assay sample responses. The methods can also include providing mitigation recommendations to reduce levels of anaerobic sulfate-reducing bacteria in a source of fluid for the fluid test sample based on a combination of information determined from the integrative spectral comparisons.

To better understand and appreciate the surprisingly flexible advantages and benefits of the innovative technologies disclosed herein for various applications, it is helpful to understand certain environments where SRB have a significant detrimental impact.

H₂S can be corrosive, malodorous, and/or toxic. H₂S can be produced through bacterial decomposition of organic matter in environments and processes such as sewers, wastewater treatment plants, water wells, oil wells, and petroleum refineries. H₂S can also form metal sulfide scales that damage equipment.

In certain residential contexts, such as second homes or cabins, temporarily unoccupied residences, or similar circumstances where fluid is allowed to linger in an anaerobic environment, sulfate-reducing bacteria (SRB) cause a specific type of corrosion known as microbiologically influenced corrosion (MIC) in metallic fluid containers and conduits. This form of corrosion occurs as follows:

Sulfate-reducing bacteria (SRB) cause a specific type of corrosion known as microbiologically influenced corrosion (MIC) in water heaters and other metal structures. This form of corrosion occurs as follows:

Anaerobic Environment Creation—SRB thrive in anaerobic (oxygen-free) environments, often found in stagnant water sections of water heaters or pipelines. These conditions are ideal for the growth and metabolism of SRB.

Sulfate Reduction—The anaerobic SRB reduce sulfates present in the water to hydrogen sulfide, chemical formula H₂S as depicted in illustration shown in the upper lefthand portion of FIG. 1. This process involves the anaerobic SRB 102 consuming sulfate as an electron acceptor in the absence of oxygen and producing H₂S 134 as a byproduct.

Metal Attack—The produced H₂S 134 is highly corrosive to metals, including steel and iron, commonly used in water heaters, tank, pipes and so forth. H₂S 134 can lead to the formation of iron sulfide on the metal surfaces, which is a weak and porous layer, providing little protection against further corrosion. Additionally, the production of H₂S 134 leads to the formation of sulfuric acid under certain conditions, further accelerating the corrosion process. Corrosion that is influenced by SRB may be referred to as microbial influenced corrosion or MIC. Also depicted in illustration shown in the upper lefthand portion of FIG. 1 is a cross-sectional view of metal with MIC 148 in the form of a pit on the surface.

Pitting Corrosion—MIC, particularly from SRB, results in pitting corrosion, a localized form of corrosion that leads to the creation of small, deep pits on the metal surface. These pits can become initiation points for cracks and ultimately lead to structural failure if not addressed.

Galvanic Corrosion—In systems where different metals are in contact, the presence of SRB also exacerbates galvanic corrosion. The metabolic byproducts of these anaerobic SRB 102 can change the local electrochemical conditions, enhancing the galvanic cell formation between dissimilar metals, leading to increased corrosion rates of the more anodic material.

Controlling SRB-induced corrosion involves maintaining water chemistry to prevent SRB growth, using biocides to control bacterial populations, employing corrosion inhibitors, and designing systems to minimize stagnant areas where anaerobic conditions might develop. As explained in more detail in the following representative examples, anaerobic SRB 102 and the various forms of MIC 148 that they induce can have detrimental effects in a wide range of environments.

In FIG. 1, a residential water heater 146a is depicted as a representative example illustration of microbial influenced corrosion (MIC) 148, which if left unaddressed could cause primary damage to the water heater itself. Water heaters, especially those that are not regularly maintained or are set to lower temperatures, can harbor SRB growth. The anaerobic conditions and presence of sulfate in the water supply can lead to the production of H₂S, which can be released into the air when hot water is used, Moreover, MIC 148 can lead to catastrophic failure of a metal tank or pipe, causing fluid (for example, water damage) which in residential settings such as second homes, cabins, residences for sale, etc., could cause substantial damage to the residential structure, fixtures, and contents. Although the residential water heater 146a is selected as a symbol illustration of residential impact of SRB, other residential facilities can also be affected. Septic systems and sewage pipes are common in residential areas for waste management. SRB thrive in these anaerobic, sulfate-rich environments, producing H₂S as a by-product. If these systems are not properly maintained, H₂S gas can accumulate and potentially seep into homes, posing health risks. Basements and crawl spaces can become areas of concern if they are poorly ventilated and if there is water intrusion. These conditions create a favorable environment for SRB.

The H₂S gas produced can accumulate in these confined spaces, leading to exposure risks for residents. Well water systems, particularly those in areas with high sulfate content in groundwater, can be susceptible to SRB activity. H₂S produced by these bacteria can lead to sulfur odors and potential health risks when the water is used for drinking, cooking, or bathing. Drainage systems, including sump pits, can collect organic matter and water, creating an anaerobic environment conducive to SRB growth. The H₂S gas produced can enter living spaces if these areas are not properly sealed or ventilated. Plumbing systems in rarely used areas of a home, such as guest bathrooms, can have stagnant water that serves as a breeding ground for SRB. This can lead to the production of H₂S, particularly when the water is agitated after a period of non-use.

Exposure to H₂S and SRB metabolites can cause a range of health issues, from mild irritation of the eyes and respiratory system to more severe effects such as headaches, nausea, dizziness, and in extreme cases, respiratory distress or death due to high concentrations. It's important for homeowners to be aware of these risks and to take preventive measures, such as ensuring proper ventilation, regular maintenance of water and waste systems, and monitoring for signs of SRB activity or H₂S presence.

Regular monitoring and maintenance are crucial to detect and mitigate the early stages of MIC. However, most residential homeowners or caretakers an unaware of the potential problems of SRB until it is too late. Reliable and easy-to-use SRB detection techniques are either unavailable for residential fluid testing or can provide false positive results causes undue stress and/or expense or false negative results masking early detection of a potential problem. As much as many residential owners are unaware of and lack access to kits, systems, and methods for reliable monitoring for the presence of SRB, many industrial owners are all too familiar with the problem and yet continue to rely on antiquated or only partially effective detection techniques.

Because SRB induce microbiologically influenced corrosion that significantly impacts various industries by accelerating corrosion in pipelines, storage tanks, and other infrastructure, the inventors of the technologies disclosed herein have developed a system 100 that is portable for field use for rapid multimodal detection of anaerobic SRB 102 in a fluid test sample 104 via integrative spectral comparison 106 so that viable anaerobic SRB 102 can be reliably detected and appropriate mitigation recommendations can be provided before corrosive damage occurs.

FIG. 1 also depicts oil and gas processing facilities 146b with corroded and structurally damaged areas 150 of petroleum product storage tanks symbolizes the devastating impact that SRB-related MIC can have of the oil and gas industry and on other industries most affected by SRB-induced MIC.

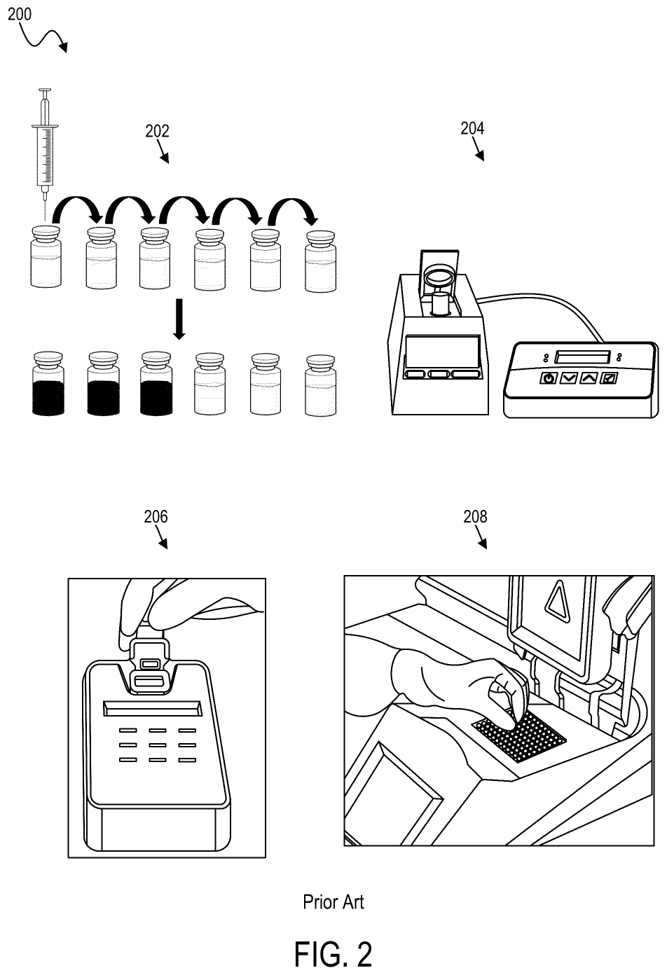

Oil and Gas Industry—SRB pose significant challenges in the oil and gas sector, impacting pipelines, storage tanks, and offshore platforms. The combination of water, hydrocarbons, and fluctuating temperatures provides ideal conditions for SRB proliferation. The total cost of corrosion in this industry is estimated at several billion dollars annually, with MIC accounting for a substantial share of these costs. As further elaborated in FIG. 2, reliably testing for SRB has been a persistent challenge in the oil and gas industry, with effective solutions remaining difficult to achieve. Because other industries also lack effective solutions, they too suffer the effects of SRB and MIC.

Water and Wastewater Treatment—SRB can thrive in sewage pipelines, treatment plants, and storage tanks, causing corrosion that can lead to leaks and structural failures. The cost of corrosion in the water and wastewater industry is also in the billions of dollars annually, with MIC playing a substantial role.

Maritime Industry—Ships, offshore structures, and port facilities are susceptible to SRB-induced MIC, especially in ballast tanks and areas where seawater is used or stored. Corrosion costs in the maritime industry are significant, with MIC contributing to maintenance, repair, and dry-docking expenses. The total cost is again in the range of billions globally, with SRB being a notable factor.

Power Generation—Cooling systems, especially in nuclear power plants where water is used as a coolant, can suffer from SRB-related MIC, affecting the integrity of cooling pipes and systems. The specific costs associated with SRB-related MIC in this sector are not frequently detailed but contribute to the broader corrosion management expenses within the power generation industry.

Globally, the estimated cost of corrosion is over $2.5 trillion, which is about 3-4% of the GDP of industrialized countries, with MIC accounting for a significant fraction of these costs. SRB-related MIC is a critical component of these expenses, especially in industries where moisture and sulfate conditions are prevalent. The actual costs include direct expenses related to repairs and replacements, as well as indirect costs like production losses, environmental impacts, and safety concerns.

FIG. 2 (Prior Art) is an overview 200 illustrating existing approaches for assessing indications of SRB in fluid test samples.

A serial dilution culturing approach 202, in use since the 1950s, employs sterile sample bottles with SRB detection media, requiring aseptic sample injection growth observations over an extended incubation of 7 to 28 days to estimate bacterial populations, depending on environmental factors such as temperature. This is cumbersome for field testing and prone to bacterial underestimation.

An ATP photometry approach 204, adopted by various users in the 1980s, measures adenosine triphosphate to determine microbial biomass. Some results may be obtained in about 1 hour from fluid test sample collection, but the ATP photometry approach 204, cannot differentiate bacterial species or assess specific SRB activity.

A filtered enzyme substrate dye approach 206, introduced in the 1990s, captures bacteria on membranes and uses enzyme substrate reactions to produce a detectable signal but can require from about 0.5 to about 12 hours depending on incubation which often leads to inaccuracies. Furthermore, the filtered enzyme substrate dye approach 206 relies on complex computations for quantitative analysis with reduces its effectiveness in field scenarios. Additionally, it does not concurrently evaluate H₂S levels, thereby failing to provide integrative insights.

A molecular microbiological methods approach 208, primarily adopted by laboratories since the 2000s, leverages advanced techniques such as DNA/RNA amplification and sequencing to provide relatively precise bacterial analysis. However, the molecular microbiological methods approach 208 require about 5-7 days for sequencing and also required specialized equipment and technical expertise which renders molecular microbiological methods approach 208 expensive, prone to delays, and unsuitable for field applications. Furthermore, while the molecular microbiological methods approach 208 can be precise regarding the microbiology of SRB, this approach is ill-suited for integrative analysis because it does not provide concurrent assessment of H₂S levels in the field.

This section provides further details about the history and usage of these methods as well as their limitations for comparison with the new and innovative systems, kits, and methods disclosed in the present application.

Because of the enormous global costs of SRB-related MIC, the Association for Materials Protection and Performance (AMPP) formed from the merger of Society for Protective Coatings and National Association of Corrosion Engineers (NACE International) which purports to be the largest technical society dedicated to the mitigation and control of corrosion has publish a Standard (NACE TM0194-2014) for Field Monitoring of Bacterial Growth in Oil and Gas Systems. The NACE TM1094-2014 Standard, which as of the filing date of this application is the most current standard intended for use by technical field and service personnel the describes field methods for estimating bacterial populations, including sessile bacterial populations, commonly found in oilfield systems. Sampling methods are emphasized and media for enumerating common oilfield bacteria are described.

Yet the NACE TM1094-2014 standard, acknowledges limitations and/or shortcomings with existing techniques for monitoring of bacterial growth (particularly SRB) in oil and gas systems, specifically stating that “This standard describes field test methods for estimating bacterial populations commonly found in oil and gas systems. Although these techniques have been successful in the oil field, they are not the only methods that are used. Regardless of the method chosen, all techniques should be applied in a consistent manner. It should be recognized that transportation of samples from the field before analysis can significantly change the viability of the bacteria and therefore, whenever practical, analysis should be initiated in the field. It is not the intent of this standard to exclude additional techniques that can be proved useful. However, caution should be exercised with any technique that is at variance from those outlined here.”

For field testing, the NACE TM1094-2014 describes a serial dilution culturing approach 202 for estimating sulfate-reducing bacteria (SRB) levels in a fluid test sample. This serial dilution culturing approach 202 uses sterile sample bottles containing anaerobic, sterilized SRB detection media, to which diluted samples are aseptically injected. In the context of field testing, many requirements of the serial dilution culturing approach 202 are cumbersome and/or difficult to perform with other steps.

For example, a section of the NACE TM1094-2014 standard on sampling bottles states that “To minimize changes, the sample should be analyzed without delay, preferably on site. If a delay of more than one hour is unavoidable, it should be noted that errors in bacterial population estimates could still result. If the delay is greater than 48 h, the sample should be refrigerated. If samples are not immediately analyzed on site, the containers must be sealed and placed in a cooler or refrigerator to reduce the bacterial metabolism until testing is performed. Testing should be performed only after the refrigerated samples have been allowed to warm up to ambient temperature. The time delay occurring between sampling and analysis should be held constant for all testing. For example, if some samples are normally analyzed 4 h after collection, all samples should be held for 4 h before testing.”

Later, in a section concerning incubation, the standard states that “The proper incubation temperature is critical to growing bacteria removed from the field system. Therefore, the incubation temperature must be within ±5° C. (±9° F.) of the recorded temperature of the water when sampled. This incubation temperature must be recorded. Because oilfield bacteria can grow in produced fluids at temperatures of 80° C. (176° F.) or higher, special incubation procedures can be required when high-temperature fluids are encountered.”

The person performing the field test is instructed to “Estimate bacteria numbers using Table A (depicted below). However, it must be noted that using this table is simplistic. Estimating bacterial populations by the serial dilution method is a subject for statistical analysis. The more replicate samples done, the tighter the statistical distribution, and the more precise the estimate. With the testing prescribed in this standard, the ranges of bacterial populations shown in Table 1 are actually too narrow. Adding to the confusion is the fact that bacterial media inherently underestimate bacterial populations. However, by convention, the values reported in Table 1 are considered acceptable for oilfield situations.”

Prior Art Table A- Serial Dilution Results
Interpretation -(NACE TM0194-2014)

| | | Growth (+) | Reported |
|---|---|---|---|
| No. of | Actual Dilution of | Indicated | Bacteria per |
| Positive Vials | Sample | Bacteria per mL | mL |
| 1 | 1:10 | 1-9 | 10 |
| 2 | 1:100 | 10 to 99 | 100 |
| 3 | 1:1000 | 100 to 999 | 1,000 |
| 4 | 1:10,000 | 1,000 to 9,999 | 10,000 |
| 5 | 1:100,000 | 10,000 to 99,999 | 100,000 |
| 6 | 1:1,000,000 | 100,000 to 999,999 | 1,000,000 |

The NACE TM0194-2014 standard also describes what it terms “non-media based field methods.” One representation example of a non-media based field method is an adenosine triphosphate (ATP) photometry approach 204 as further depicted in FIG. 2. ATP is present in all living cells and is involved in all processes that require energy, but because it rapidly disappears on cell death, thus, although the adenosine triphosphate (ATP) photometry approach 204 can give an indication of the viable biomass present in living organisms, ATP photometry does not provide bacterial speciation in the sample (no differentiation can be determined), it can only be used to assess gross microbial contamination and does not distinguish between bacteria types and therefore cannot be used to determine whether bacteria present in a sample present a risk of producing H₂S.

Another conventional method is a filtered enzyme substrate dye approach 206. The filtered enzyme substrate dye approach 206 involves capturing bacteria from the sample on small particles containing antibodies that specifically bind to adenosine-5-phosphosulfate (APS) reductase passing a fluid test sample through a porous membranes or microscale filters and capturing bacteria from the sample on a filter membrane. The captured bacteria are then exposed to a solution including an enzyme substrate dye. Both viable and non-viable bacteria can be captured releasing a phosphorescent dye, thus potentially indicating an incorrect estimate. Additionally, the filtered enzyme substrate dye approach 206 requires complex computations and quantitative analysis, does not detect H₂S, and can still suffer from longer incubation periods than are desirable.

The NACE TM1094 standard also describes “methods below are laboratory based and provide further definition to the bacterial populations examined but are not normally associated with field techniques.” These are referred to herein as the molecular microbiological methods (MMM) approach 208, which involves extracting, amplifying, quantifying and/or sequencing bacterial DNA and RNA. The molecular microbiological methods (MMM) approach 208 typically requires highly trained technicians and large investments in equipment, supplies, and infrastructure. The molecular microbiological methods (MMM) approach 208 can also require laboratory testing periods of multiple days or weeks to get results.

As evidenced above, the closest prior art for assessing sulfate-reducing bacteria (SRB) includes four methods: the serial dilution method, ATP photometry, filtered enzyme substrate dye method, and molecular microbiological methods (MMM). The serial dilution method, described in the NACE TM0194-2014 standard, involves aseptic dilution of fluid samples into sterile bottles containing SRB detection media. Only partially effective, it requires prolonged incubation periods of 7-10 days, is highly dependent on controlled environmental factors, and is subject to statistical variability and bacterial underestimation.

In contrast, the claimed subject matter, which is described in more detail in the sections that follow, achieves concurrent detection of H₂S and anaerobic SRB enzymatic/metabolic activity within 20-30 minutes, offering significantly reduced time to results, enhanced accuracy through direct biomarker detection, and field compatibility without the need for extensive incubation facilities or refrigerated transportation. An integrated assay kit includes reagents formulated to detect H₂S and the reagents formulated to detect anaerobic SRB enzymatic/metabolic activity using oxygen scavengers to help maintain an anaerobic environment for performing an integrative spectral comparison of spectral parameters indicative of both H₂S and anaerobic SRB enzymatic/metabolic activity. These innovative advancements facilitate rapid, field-usable, comprehensive quantitative and qualitative analysis and provide a wide range of field usable options including a compact assay reader, a mini assay reader, and an advanced spectrometry assay reader. Thus, the technologies disclosed herein significant analytical measurement advantages in efficiency and accuracy in performing integrative spectral comparisons, and they address critical limitations of the serial dilution method which is still widely used.

The ATP photometry approach 204 measures adenosine triphosphate (ATP) as an indicator of viable biomass but does not differentiate bacterial species or specifically detect SRB or H₂S. Moreover, the ATP photometry approach 204 does utilize rapid, easy-to-use assay sample readers with tangible spectral reference standards for rapid integrative spectral comparison in the field. The claimed subject matter overcomes these limitations by precise integrative spectral comparison of multimodal detection of H₂S and anaerobic SRB enzymatic activity, providing for comprehensive analysis. Unlike ATP photometry, which often requires laboratory equipment and provides only gross microbial contamination assessments, the innovative technologies of the present disclosure provide portable, rapid, and field-compatible integrative analysis to deliver timely and accurate actionable data.

The filtered enzyme substrate dye approach 206 captures bacteria on membranes and uses substrate dyes to indicate enzyme activity. However, it suffers from non-specificity, long incubation periods, and inaccuracies due to non-specific enzyme activity. By contrast the technologies of the present disclosure provide significantly advantageous solutions by directly targeting with reagents that include oxygen scavengers and are formulated to concurrently detect H₂S and anaerobic SRB enzymatic/metabolic activity, enabling accurate detection within 20-30 minutes. The various assay readers 108a, 108b, 108c disclosed in the present application further facilitate concurrent integrative spectral comparison of levels of H₂S and anaerobic SRB with easy-to-use apparatuses and protocols make the disclosed technologies far more practical and accurate for field applications compared to the complex filtration procedures of the prior art.

The Molecular microbiological methods (MMM) approach 208 involves advanced techniques like DNA/RNA amplification and sequencing to analyze bacterial populations. Although highly detailed, MMM requires specialized equipment, trained technicians, and extended testing periods ranging from days to weeks, making it unsuitable for rapid field applications.

By contrast, the comprehensive and flexible solutions disclosed in the present application, by contrast, provide real-time results on-site within minutes. The systems, apparatuses, and kits disclosed in the present application are accordingly significantly more cost-effective and practical than MMM, eliminating the need for sophisticated infrastructure while delivering timely actionable data and/or optimized mitigation recommendations.

In summary, as will be apparent from the description and working examples that follow, the disclosed subject matter provides numerous new and surprisingly effective yet simple and efficient advantages over all four prior art approaches by combining rapid detection, enhanced accuracy, field compatibility, and integrative multiparameter analysis. These improvements breakthrough the limitations of existing methods, delivering new and significant advancements in SRB detection technology.

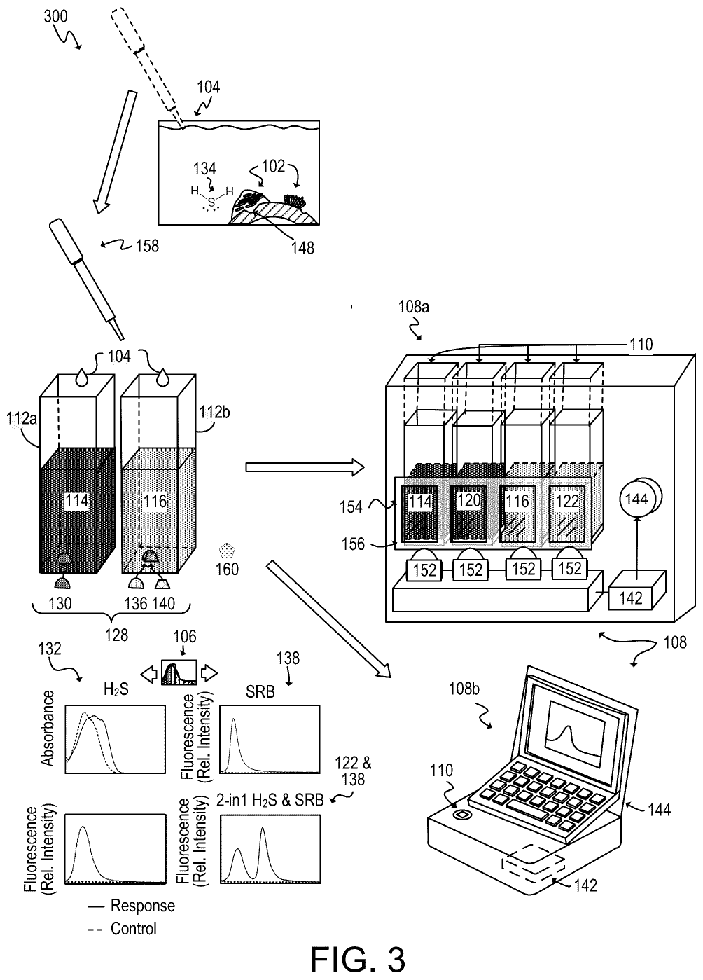

FIG. 3 is a schematic block diagram illustrating a system 300 for field use in rapid multimodal detection of anaerobic SRB 102 in a fluid test sample 104 via integrative spectral comparison of two or more assay samples 114, 116 prepared using a kit 128 with first reagents 130 formulated to detect H₂S 134 and second reagents 136 formulated to detect anaerobic SRB with oxygen scavengers 140 to maintain an anaerobic environment, according to one or more aspects of the present disclosure.

In some implementations, the assay reader 108 is an compact assay reader 108a that includes one or more windows 154 through which one or more spectral parameters of the first set of spectral parameters 132 and/or the second set of spectral parameters 138 pertaining to the first assay sample 114 and/or the second assay sample 116 are determinable, for example by direct comparison with a tangible spectral reference standard such as the first spectral reference standard 120 and the second spectral reference standard 122. The windows 154 may include optical filters 156 that facilitates comparison of the spectral response of the assay sample 114, 116 with the respective spectral response of the spectral reference standard 120, 122 by filtering out excitation wavelengths so that for example, fluorescence spectral responses have less interference from UV excitation when compared through the optical filters 156.

In certain examples, the assay reader 108 is an advanced spectrometry assay configured to detect and analyze the first set of spectral parameters and the second set of spectral parameters pertaining of the integrative assay sample concurrently, and/or using multiplexing. Further details about such implementations are provided below with respect to FIG. 4.

Examples of the present disclosure include a kit 128 formulated to detect active SRB in an assay sample 114, 116, 118. In some examples, the kit 128 includes one or more first reagents 130 and one or more second reagents 136 formulated to indicate, via spectral parameters, one or both of a level of H₂S and/or microbial activity in a fluid test sample 104. In certain examples, the kit 128 is formulated to indicate the levels of H₂S and microbial activity in the fluid test sample 104 simultaneously and/or nearly simultaneously. As such, various examples of the present disclosure help to confirm causation of H₂S by indicating whether the H₂S is linked to microbial activity in the fluid test sample 104. In one or more examples, an aliquot of the fluid test sample 104 is added to a one or more assay containers 112 (for example, via a fluid handler 158) to be exposed to the first reagents 130 and/or second reagents 136, thereby forming a first assay sample 114, a second assay sample 116, or a combination assay sample 118.

As used herein, the term “spectral parameters” includes any parameters which can be observed visually, via spectroscopy, and/or using colorimetry. In various examples, a first set of spectral parameters includes parameters exhibited by a compound produced as a result of a reaction between one or more first reagents 130 and H₂S in the fluid test sample 104. In some examples, a second set of spectral parameters includes a parameter exhibited by a compound formed as a result of a reaction between the one or more second reagents 136 and microbial enzymes within the fluid test sample 104. In certain examples, spectral parameters include fluorescence and/or color of a component produced as a result of a reaction between at least one of the reagents and at least one component with the fluid test sample 104 (for example, H₂S and/or microbial enzymes). In one or more examples, the spectral parameters include a wavelength range. In some examples, the first set of spectral parameters and the second set of spectral parameters overlap and/or are identical.

In certain examples, the first reagents 130 are formulated to indicate, via a first set of spectral parameters, a level of hydrogen sulfide (H₂S) dissolved in a fluid test sample 104. In various examples, the one or more first reagents 130 include colorogenic and/or fluorogenic reagents that are sulfide-reactive. In one or more examples, the one or more second reagents 136 are formulated to indicate, via a second set of spectral parameters, a level of enzymatic activity of one or more species of anaerobic sulfate-reducing bacteria in the fluid test sample 104. In some examples, the one or more second reagents 136 are formulated to not react with non-bacterial components in the fluid test sample 104. In certain examples, the second reagents 136 include colorogenic and/or fluorogenic reagents. In some examples, the one or more first reagents 130 are formulated to detect H₂S from both active or past sulfate-reducing bacteria in the fluid test sample 104. In such examples, the near-simultaneous detection of microbial activity via the one or more second reagents 136 helps to confirm that the detected H₂S results from active SRB and not from past SRB in the fluid test sample 104.

As used herein, the term “colorogenic reagent” includes reagents formulated to indicate at least one of a level of H₂S and/or a level of microbial activity in a fluid test sample 104 via a spectral parameter that results from reaction between first reagents 130 and/or second reagents 136 and the component(s) being detected (i.e., H₂S and bacterial enzymes). In one or more examples, the spectral parameters are produced by a compound that results from the reaction.

In some examples, the spectral parameter includes a color of light emitted by the compound, a change in the color exhibited by the fluid test sample 104, and/or a wavelength range of peak absorbance for the fluid test sample 104 as a result of the compound. As used herein, the terms “colorogenic” and “colorigenic” can be used interchangeably.

As used herein, the term “fluorogenic reagent” includes reagents formulated to indicate at least one of a level of H₂S and/or a level of microbial activity in a fluid test sample 104 via a spectral parameter that results from reaction between first reagents 130 and/or second reagents 136 and the component(s) being detected (i.e., H₂S and bacterial enzymes). In various examples, the spectral parameter is produced by a compound that results from the reaction. In some examples, the spectral parameter includes a fluorescence of the compound and/or a change in the fluorescence exhibited by the fluid test sample 104.

In certain examples, the kit 128 includes one or more oxygen scavengers 140 combined with the one or more second reagents 136 and formulated to, upon contact with the fluid test sample 104, maintain an anaerobic environment for the one or more species of anaerobic sulfate-reducing bacteria. In some examples, the one or more oxygen scavengers 140 help to reduce oxygen in the fluid test sample 104, thus inhibiting anaerobic respiration and reducing other oxidative stresses. In one or more examples, the one or more oxygen scavengers 140 include sodium dithionite and/or sodium sulfite. In various examples, the kit further includes the oxygen scavenger 140 comprising reduced glutathione with IUPAC name (2S)-2-amino-5-[[(2R)-1-(carboxymethylamino)-1-oxo-3-sulfanylpropan-2-yl]amino]-5-oxopentanoic acid, that in combination with sodium dithionite, synergistically enhances oxygen-scavenging properties of the combination.

In one or more examples, the first reagents 130 and/or second reagents 136 are lyophilized. In such examples, first reagents 130 and/or second reagents 136 can be added to the fluid test sample 104 without introducing additional fluids to the fluid test sample 104. Such examples can help to preserve ionic conditions of the fluid test sample 104 and/or help to reduce effects of osmotic shock to micro-organisms such as anaerobic SRB within the fluid test sample 104.

In various examples, the kit 128 further includes one or more buffering agents 160. In certain examples, the one or more buffering agents 160 are formulated to help the fluid test sample 104 resist changes in pH. In one or more examples, the one or more buffering agents 160 include at least one of the following: an acetate buffer, a phosphate buffer, a 2-(N-morpholino)ethanesolfonic acid (MES) buffer, and/or any combination thereof. In some examples, the buffering agents 160 help to ensure that the reaction product of the H₂S and the one or more first reagents 130 exhibits a desired color. In certain examples, the one or more buffering agents 160 are added to the fluid test sample 104 in the same assay container 112a holding the one or more first reagents 130. In various examples, the one or more buffering agents 160 can help to maintain the pH of the one or more first reagents 130 within a particular range. In some examples, the pH range is not less than 7.4 and not greater than 8.5. For example, phosphate buffer or Tris-HCl buffer are examples of buffering agents 160 suitable for maintaining the pH in the optimal range for 5-[(3-carboxy-4-nitrophenyl)disulfanyl]-2-nitrobenzoic acid reactions with H₂S in aqueous samples, provided that their potential interactions with assay components are considered.

In one or more examples, the kit further includes a spectral reference standard 120, 122, 124. In certain examples, the spectral reference standard 120, 122, 124 is made to indicate, via at least one of the first set of spectral parameters and the second set of spectral parameters corresponding to wavelengths associated with reaction of the one or more first reagents 130 with at least one of a baseline level of H₂S and wavelengths associated with reaction of the one or more second reagents 136 with a baseline level of enzymatic activity of the one or more species of anaerobic sulfate-reducing bacteria.

In some examples, the spectral reference standard 120, 122, 124 has spectral characteristics corresponding to one or more parameters of the first set of spectral parameters and/or the second set of spectral parameters. In one or more examples, the spectral parameters include a colorimetric parameter. In various examples, the spectral reference standard 120, 122, 124 exhibits a baseline color corresponding to the colorimetric parameter. In certain examples, the spectral reference standard 120, 122, 124 includes a one or more dyes corresponding to predetermined spectral parameters. In some examples, the spectral reference standard 120, 122, 124 includes a solid casting resin containing a dye and/or a solid casting compound impregnated with a dye. In one or more examples, the spectral parameters include a color comparison to the spectral reference standard 120, 122, 124 indicating the presence of H₂S and/or microbial activity in the fluid test sample 104. In some examples, the spectral reference standard 120, 122, 124 includes a dye of a lighter shade of a particular color (for example, a light yellow), and the spectral parameter includes a darker shade of that same color (for example, a darker yellow). In various examples, the dye of the spectral reference standard 120, 122, 124 is a fluorescence dye. In certain examples, the spectral reference standard 120, 122, 124 is formulated to exhibit a baseline parameter in response to excitation by an excitation source 152 (for example, via blue light).

In some examples, the system 100 includes one or more assay containers 112 that are configured to receive at least a portion of the fluid test sample 104 and one or more of the first reagent 130 and the second reagent 136. In various examples, the kit 128 includes assay containers 112, each one or more assay containers 112 holding at least one of: the one or more first reagents 130, the one or more second reagents 136, oxygen scavengers 140, and/or buffering agents. In certain examples, the fluid test sample 104 is added to the one or more assay containers 112 after the container 112 has received at least one of the one or more first reagents 130 and/or the one or more second reagents 136.

In one or more examples, a single one or more assay containers 112 of the one or more assay containers is configured to receive the one or more first reagents 130 and the one or more second reagents 136 concurrently and serves as a single reaction vessel for first reagents 130 and/or second reagents 136. As such, in certain examples, detection of H₂S and microbial enzymatic activity can be accomplished in a single reaction in one assay container 112.

In other examples, the detection of H₂S via the first reagent 130 takes place using a first assay container 112a, and the detection of microbial activity via the second reagent 136 takes place using a second assay container 112b.

As used herein, the term “assay container” can refer to any container suitable for holding one or more of first reagents 130, second reagents 136, and oxygen scavengers 140, which when combined with the fluid test sample 104 form one or more assay samples 114, 116.

In some examples, the term assay containers 112 can be used to refer to one or more of the following: a vessel, a cuvette, a tube, and/or any combination thereof. In certain examples, the one or more assay containers 112 are disposable.

In various examples, the one or more first reagents 130 are formulated such that the fluorescence of a component yielded by the reaction of the one or more first reagents 130 with the H₂S is excited by exposure to light and causes fluorescence of the component yielded by the reaction of the one or more second reagents 136 with the microbial enzymes. In one or more examples, the first set of spectral parameters and/or the second set of spectral parameters includes a fluorescence of above a particular wavelength in response to excitation with light. In certain examples, the particular wavelength is 500 nm in response to excitation with UV light.

In some examples, the one or more first reagents 130 and/or the one or more second reagents 136 are formulated to exhibit the first set of spectral parameters and/or the second set of spectral parameters within a predetermined incubation period of the fluid test sample 104 being introduced to the first reagents 130 and/or the second reagents 136 via the one or more assay containers 112. In various examples, the predetermined incubation period is approximately one minute. In certain examples, the incubation period is approximately 8 hours.

In certain examples, the kit 128 includes ascorbic acid, which can be added to the fluid test sample 104 in the relevant assay container 112a, 112b, 112c to help reduce dissolved H₂S within the fluid test sample 104 in the assay container.

As shown in Table 1, in various examples, the one or more first reagents 130 may include a colorogenic reagent, examples of which are listed in Table 1.

TABLE 1
Colorigenic H₂S Detection Reagents

| | | | Analysis |
|---|---|---|---|
| ID | Reagent | | Range |
| Num. | Name (IUPAC) | Chemical Structure | (nm) |
| T1- Rgt1 | 5-[(3-carboxy-4- nitrophenyl) disulfanyl]-2- nitrobenzoic acid | 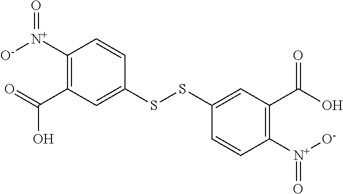 [SMILES: O=C(O)c1cc(SSc2ccc([N+](=O)[O-])c(C(=O)O)c2)ccc1[N+](=O)[O-]] | 380-440 |
| T1- Rgt2 | p-[(E)-4-Hydroxy-1- naphthylazo] benzenesulfonic acid | 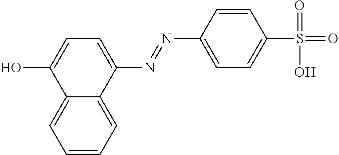 [SMILES: O=S(=O)(O)c1ccc(/N=N/c2ccc(O)c3ccccc23)cc1] | 550-590 |
| T1- Rgt3 | 4-Amino-6- [(E)-(E)-1′b-(8- amino-1-hydroxy- 5,7-disulfo-2- naphthylimino)- 3,3′-dimethyl-1′a- benzidineyl- ideneamino]- 5-hydroxy-1,3- naphthalenedisulfonic acid | 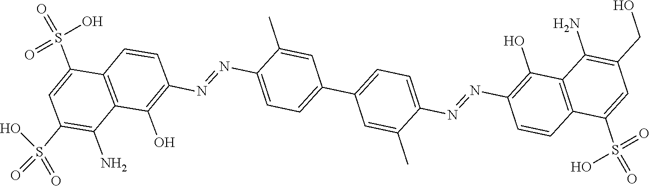 [SMILES: Cc1cc(-c2ccc(/N=N/c3ccc4c(S(=O)(=O)O)cc(S(=O)(=O)O)c(N)c4c3O)c(C)c2)ccc1/N=N/c1ccc2c(S(=O)(=O)O)cc(CO)c(N)c2c1O] | 640-690 |
| T1- Rgt4 | 4-N,4-N- dimethylbenzene- 1,4-diamine & ferric oxide | 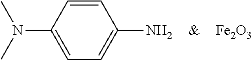 | 630-700 |
| T1- Rgt5 | 4-(4-amino-3,5- dimethylphenyl)-2,6- dimethylaniline & silver nitrate | 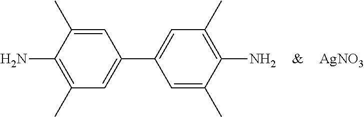 [SMILES: Cc1cc(-c2cc(C)c(N)c(C)c2)cc(C)c1N ; "AgNO3"] | 630-680 |

In some examples, the first reagents 130 are formulated to produce a visible spectrum wavelength change in the fluid test sample 104 upon reacting with H₂S in the fluid test sample 104. In some examples, the one or more first reagents 130 include a colorogenic reagent formulated to change in color from yellow to red and/or from blue to violet when reacting with H₂S.

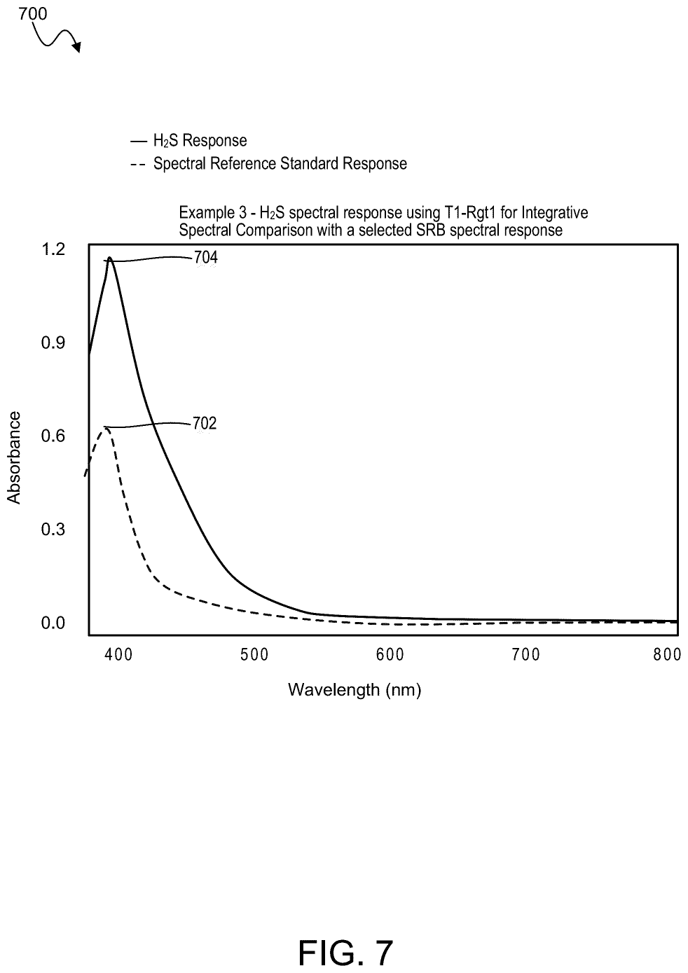

Accordingly, in certain implementations, the one or more first reagents are formulated to include p-[(E)-4-Hydroxy-1-naphthylazo]benzenesulfonic acid for enhancing a spectral comparison of a reaction of H₂S with the p-[(E)-4-Hydroxy-1-naphthylazo]benzenesulfonic acid, where the fluid test sample before contacting the one or more first reagents produced a yellow baseline color and/or a target wavelength to be detected is in a range of about 550 nanometers (nm) to about 590 nm. More specifically, in some examples, the one or more first reagents 130 are formulated to include p-[(E)-4-Hydroxy-1-naphthylazo]benzenesulfonic acid for enhancing a spectral comparison of a reaction of H₂S with the p-[(E)-4-Hydroxy-1-naphthylazo]benzenesulfonic acid (T1-Rgt2), as shown in FIG. 7. In some examples, the p-[(E)-4-Hydroxy-1-naphthylazo]benzenesulfonic acid changes in color from yellow to red after and/or upon reacting with H₂S in the fluid test sample 104. In some examples, the fluid test sample 104, before contacting the one or more first reagents 130, has a yellow baseline color and/or a peak absorbance wavelength in a range of about 550 nm to about 590 nm.

In various examples, the one or more first reagents 130 include a colorogenic reagent, and the spectral parameter includes a wavelength of light emitted by a component produced via reaction of the H₂S in the fluid test sample 104 with the one or more first reagents 130. In some examples, the one or more first reagents 130 is formulated to produce a component by reacting with H₂S in the fluid test sample 104, and the component exhibits a particular color. In some examples, the component has a peak absorbance of approximately 412 nanometers. In some examples, the component emits light having a wavelength of not less than 570 nm and not greater than 590 nm. In some examples, the one or more first reagents 130 include 5-[(3-carboxy-4-nitrophenyl)disulfanyl]-2-nitrobenzoic acid. In some examples, the 5-[(3-carboxy-4-nitrophenyl)disulfanyl]-2-nitrobenzoic acid reacts with the H₂S in the fluid test sample 104, resulting in the production of the colorogenic component 5-mercapto-2-nitrobenzoic acid.

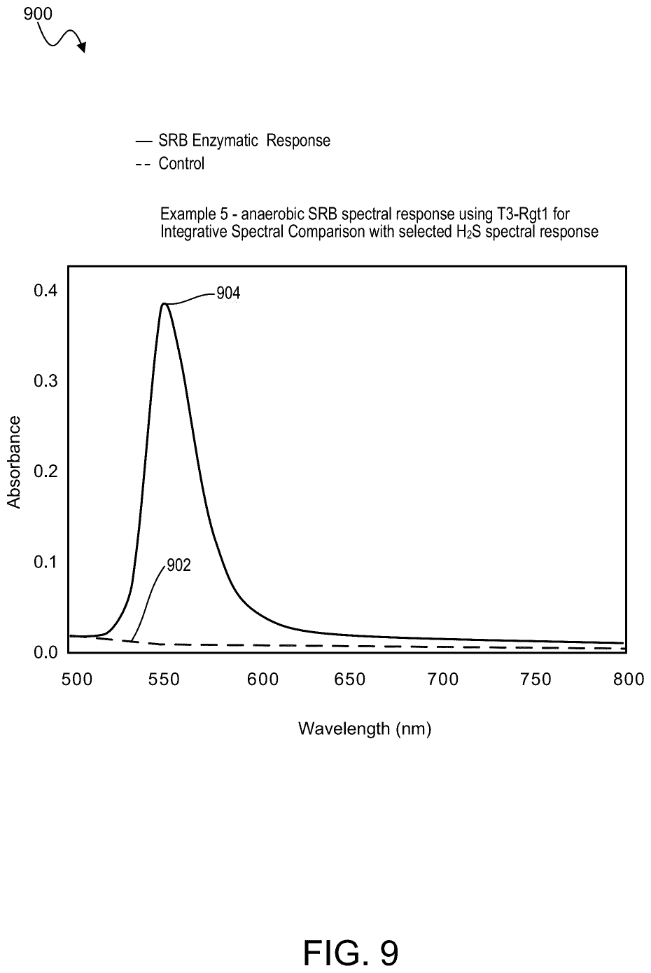

In some examples, the one or more first reagents 130 includes a reagent configured to emit light of a yellow color, or having a wavelength of not less than 570 nanometers (nm) and not greater than 590 nm. In some examples, the detection of H₂S occurs via reaction with 5-[(3-carboxy-4-nitrophenyl)disulfanyl]-2-nitrobenzoic acid, which as shown in FIG. 9, emits light having a wavelength (λₘₐₓ) of approximately 415 nm at or near its maximum absorbance.

As shown in Table 2, below, in some examples, the first reagents 130 include fluorogenic reagents. Examples of such fluorogenic reagents include, but are not limited to, the examples listed in Table 2, below.

TABLE 2
Fluorogenic H₂S Detection Reagents

| | | | Excitation | Emission |
|---|---|---|---|---|
| ID | Reagent Name | | Range | Range |
| Num. | (IUPAC) | Chemical Structure | (nm) | (nm) |
| T2- Rgt1 | 7-azido-4- methylchromen-2- one | 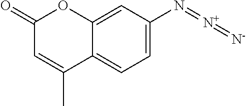 | 330-390 | 400-500 |
| T2- Rgt2 | N,N-Dimethyl[5- (azidosulfonyl)-1- naphthyl]amine | 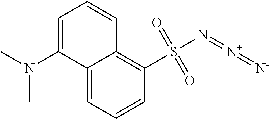 [SMILES: CN(C)c1cccc2c(S(=O)(=O)N=[N+]=[N-])cccc12] | 330-390 | 480-580 |
| T2- Rgt3 | (E)-1-(3,3- Dimethyl-1-propyl- 3H-indol-2-yl)-2-[6- (2,4- dinitrophenoxy)- 2,3-dihydro-1H- xanthen-4- yllethene | 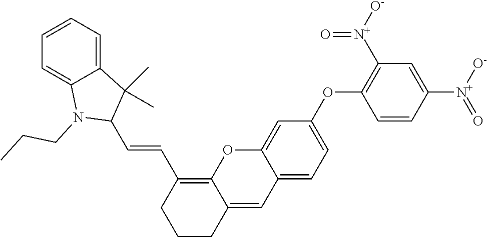 [SMILES: CCCN1c2ccccc2C(C)(C)C1/C=C/C1=C2Oc3cc(Oc4ccc([N+](=O)[O-])cc4[N+](=O)[O-])ccc3C=C2CCC1] | 630-700 | 700-780 |
| T2- Rgt4 | 5-(1,3- Benzothiazol-2-yl)- 4-(7-nitro-2,1,3- benzofurazan-4- yloxy)-3- tolualdehyde | 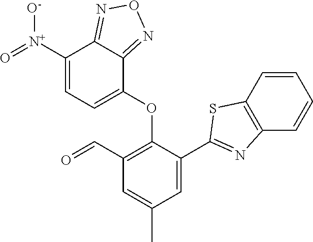 [SMILES: Cc1cc(C=O)c(Oc2ccc([N+](=O)[O-])c3nonc23)c(-c2nc3ccccc3s2)c1] | 420-480 | 500-600 |
| T2- Rgt5 | 7-{5-[p-(2,4- Dinitrophenyl- sulfonyloxy) phenyl]-2- thienyl}-4-(2- thienyl)-2,1,3- benzothiadiazole | 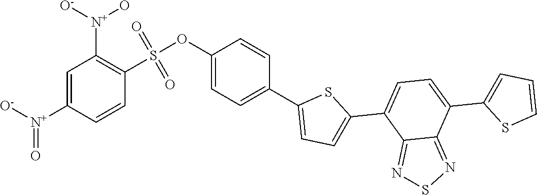 [SMILES: O=[N+]([O-])c1ccc(S(=O)(=O)Oc2ccc(-c3ccc(-c4ccc(-c5cccs5)c5nsnc45)s3)cc2)c([NH+]([O-])O)c1] | 420-480 | 500-600 |
| T2- Rgt6 | 4-{5-[4-(2,4- dinitrophenyl- sulfonyloxy)phenyl] thiophen-2-yl}-7- (thiophen-2-yl)- 2,1,3- benzothiadiazole | 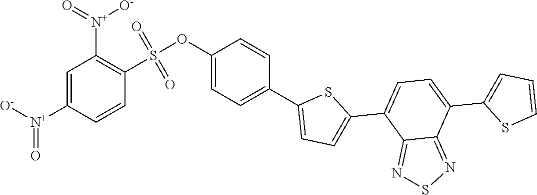 [SMILES: O=[N+]([O-])c1ccc(S(=O)(=O)Oc2ccc(-c3ccc(-c4ccc(-c5cccs5)c5nsnc45)s3)cc2)c([N+](=O)[O-])c1] | 420-480 | 500-610 |
| T2- Rgt7 | 7-Nitro-4-(7-nitro- 2,1,3- benzofurazan-4- ylthio)-2,1,3- benzofurazan | 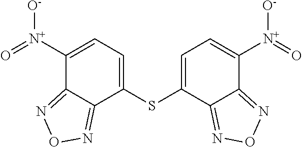 [SMILES: O=[N+]([O-])c1ccc(Sc2ccc([N+](=O)[O-])c3nonc23)c2nonc12] | 410-460 | 490-550 |
| T2- Rgt8 | 2-{4-[(E)-2-(5- Diisocyanomethyl- idene-4-isocyano- 2,2-dimethyl-2H- fur-3-yl)ethenyl]-2- bromophenoxy}-5- nitrophenylhydro- peroxyamine | 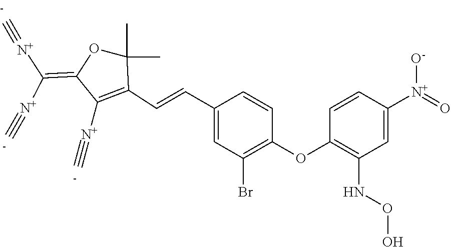 [SMILES: [C-]#[N+]C([N+]#[C-])=C1OC(C)(C)C(/C=C/c2ccc(Oc3ccc([N+](=O)[O-])cc3NOO)c(Br)c2)=C1[N+]#[C-]] | 400-470 | 550-650 |

In certain examples, the one or more first reagents 130 are formulated to include 7-Nitro-4-(7-nitro-2,1,3-benzofurazan-4-ylthio)-2,1,3-benzofurazan, which offers numerous advantages as a fluorogenic reagent for detecting H₂S 134 in fluid samples 104, particularly in applications where H₂S 134 is produced by anaerobic SRB 102. 7-Nitro-4-(7-nitro-2,1,3-benzofurazan-4-ylthio)-2,1,3-benzofurazan demonstrates significant selectivity for H₂S due to the strong nucleophilic reactivity of sulfide ions, allowing it to form a fluorescent product with minimal interference from other thiols or sulfide-containing compounds. This selectivity is particularly advantageous in complex sample matrices, such as industrial fluids or biological systems, where non-specific reactions could lead to inaccurate results.

The reaction of 7-Nitro-4-(7-nitro-2,1,3-benzofurazan-4-ylthio)-2,1,3-benzofurazan with H₂S produces a significant increase in relative fluorescence intensity, enabling the detection of even trace amounts of H₂S. This fluorogenic spectral parameter provides a high signal-to-noise ratio, ensuring both the sensitivity and accuracy required for precise quantification. Furthermore, 7-Nitro-4-(7-nitro-2,1,3-benzofurazan-4-ylthio)-2,1,3-benzofurazan reacts rapidly with H₂S, making it suitable for real-time or near-real-time monitoring applications. This rapid reaction is particularly beneficial in dynamic systems where H₂S levels may fluctuate, such as oil and gas pipelines, bioreactors, or environmental monitoring sites.

7-Nitro-4-(7-nitro-2,1,3-benzofurazan-4-ylthio)-2,1,3-benzofurazan is chemically stable under standard storage and assay conditions, ensuring consistent and reliable performance across multiple uses. Its fluorescence response is proportional to the concentration of H₂S, facilitating quantitative analysis and enabling users to obtain both qualitative and precise quantitative data. These spectral parameter are particularly valuable in industrial and environmental applications, where the extent of SRB activity must be accurately measured to mitigate risks such as microbiologically influenced corrosion or environmental contamination.

Additionally, 7-Nitro-4-(7-nitro-2,1,3-benzofurazan-4-ylthio)-2,1,3-benzofurazan exhibits versatility across various fluid types, including water, oil emulsions, and biological samples, making it suitable for diverse applications such as environmental monitoring, industrial process management, and clinical diagnostics. The reagent is highly sensitive, allowing the detection of low concentrations of H₂S, often in the micromolar range or lower, which is critical for early identification of SRB activity and proactive intervention. 7-Nitro-4-(7-nitro-2,1,3-benzofurazan-4-ylthio)-2,1,3-benzofurazan assays also require minimal specialized equipment, as detection can be performed using standard fluorescence spectroscopy or imaging tools, reducing costs and simplifying implementation.

In the context of anaerobic SRB-induced H₂S production, 7-Nitro-4-(7-nitro-2,1,3-benzofurazan-4-ylthio)-2,1,3-benzofurazan serves as an effective tool for monitoring bacterial activity, controlling corrosion, and managing safety and environmental risks. The reagent's high sensitivity, selectivity, rapid reaction kinetics, and adaptability provide a robust solution for detecting and quantifying H₂S, facilitating improved management of SRB-associated challenges in industrial, environmental, and research applications.

In some examples, the one or more first reagents 130 are formulated to include tetrasodium; 4-amino-6-[[4-[4-[(8-amino-1-hydroxy-5,7-disulfonatonaphthalen-2-yl)diazenyl]-3-methylphenyl]-2-methylphenyl]diazenyl]-5-hydroxynaphthalene-1,3-disulfonate for enhancing detection of concentrated H₂S.

In certain implementation, the one or more first reagents 130, such as for example, 7-azido-4-methylchromen-2-one are formulated to react with dissolved H₂S in the fluid test sample 104, resulting in the production of a fluorescent component, such as 4-methylcoumarin. In some examples, the fluorescent component is a component which reacts with dissolved H₂S and fluoresces in response to excitation with ultraviolet (“UV”) light (for example, UV light emitted by excitation sources 152 shown in FIG. 4). In some examples, the fluorescent component fluoresces with light of a particular color when excited with UV light. In some examples, the fluorescent component emits light having a wavelength of not less than 400 nm and not greater than 500 nm, such as around 440 nm. In some examples, the fluorescent component fluoresces in response to excitation via an excitation source emitting light of a shorter wavelength such as UV light.

In some examples, the fluorescent component emits blue light. In some examples, the UV light has a wavelength near and/or within the range of 365-370 nm. In some examples, the one or more second reagents 136 are formulated to be cleaved and/or broken down by enzymes of any sulfate-reducing bacteria with the fluid test sample 104 to yield a fluorescent component. In some examples, the one or more second reagents 136 include (6′-acetyloxy-3-oxospiro[2-benzofuran-1,9′-xanthene]-3′-yl) acetate, which is enzymatically cleaved to yield fluorescein. In some examples, fluorescein yields fluorescence of greater than or equal to 500 nm when excited with light having a wavelength of not less than 400 nm and not greater than 450 nm. In certain working examples, the one or more first reagents comprise fluorogenic reagents formulated to emit fluorescence in response to a reaction with H₂S in the in a range of wavelengths suitable to serve as an excitation source for the one or more second reagents H₂S. In such examples, fluorescent light emitted in response to the reaction of one or more first reagents serves as an excitation source for second reagents.

In some examples, as shown in Table 3, below, the second reagents 136 include fluorogenic reagents, examples of which are shown in Table 3. The “microbial enzymes” in Table 3 include examples of microbial enzymes or metabolites which the given fluorogenic reagent examples can react with.

TABLE 3
Fluorogenic Microbial Enzymatic/Metabolic Activity Detection Reagents

| | | | Excitation | Emission |
|---|---|---|---|---|
| ID | | | Range | Range |
| Num. | Reagent Name (IUPAC) | Chemical Structure | (nm) | (nm) |
| T3- Rgt1 | (6′-acetyloxy-3-oxospiro[2- benzofuran-1,9′-xanthene]-3′- yl)acetate | 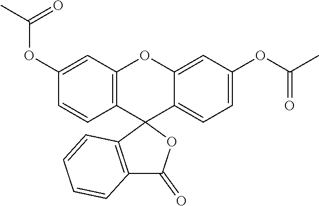 [SMILES: CC(=O)Oc1ccc2c(c1)Oc1cc(OC(C)=O)ccc1C21OC(=O)c2ccccc21] | 390-500 | 515-600 |
| T3- Rgt2 | 7-hydroxy-10- oxidophenoxazin-10-ium-3- one | 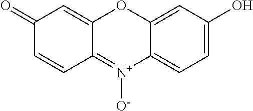 [SMILES: O=c1ccc2[n+]([O-])c3ccc(O)cc3oc-2c1] | 530-570 | 570-650 |
| T3- Rgt3 | Dipeptidyl-7-Amino-4- methylcoumarin | 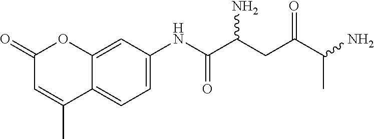 [SMILES: Cc1cc(=O)oc2cc(NC(=O)C(N)CC(=O)C(C)N)ccc12] | 340-395 | 400-500 |

In some examples, the one or more second reagents 136 formulated to indicate the level of enzymatic activity of one or more species of anaerobic sulfate-reducing bacteria in the fluid test sample 104 are selected from the group consisting of: (6′-acetyloxy-3-oxospiro[2-benzofuran-1,9′-xanthene]-3′-yl) acetate, 7-hydroxy-10-oxidophenoxazin-10-ium-3-one, Dipeptidyl-7-Amino-4-methylcoumarin, (4-nitrophenyl) phosphate, 5-Bromo-6-chloro-3-indolyl caprylate, and combinations thereof.

TABLE 4
Colorigenic Microbial Enzymatic/Metabolic Activity Detection Reagents

| ID | | | Analysis Range |
|---|---|---|---|
| Num. | Reagent Name (IUPAC) | Chemical Structure | (nm) |
| T4- Rgt1 | (4-nitrophenyl)phosphate | 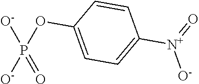 [SMILES: O=[N+]([O-])c1ccc(OP(=O)([O-])[O-])cc1] | 360-450 |
| T4- Rgt2 | 5-Bromo-6-chloro-3-indolyl caprylate | 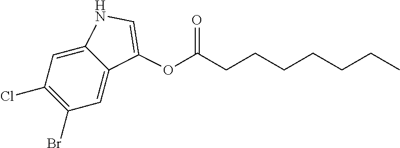 [SMILES: CCCCCCCC(=O)Oc1c[nH]c2cc(Cl)c(Br)cc12] | 570-620 |

In various examples, the kit 128 may be formulated to include certain first reagents, second reagents, oxygen scavengers, and/or other components based on the intended assay reader 108 to be used for integrative spectral comparison.

In certain implementations, the selection of colorigenic and fluorogenic reagent combinations for integrative spectral comparison of H₂S and anaerobic SRB enzymatic/metabolic activity in fluid test samples 104 is based on the specific requirements of the analysis and the capabilities of the assay reader 108 being used. In field applications where portability and rapid results are crucial, the compact assay reader 108a or mini assay reader 108c may be preferred, utilizing a combination of colorigenic reagents like p-[(E)-4-Hydroxy-1-naphthylazo]benzenesulfonic acid or 5-[(3-carboxy-4-nitrophenyl)disulfanyl]-2-nitrobenzoic acid for H₂S detection, and fluorogenic reagents such as (6′-acetyloxy-3-oxospiro[2-benzofuran-1,9′-xanthene]-3′-yl) acetate for SRB enzymatic activity.

In certain examples, such as depicted, an assay reader 108 used with a kit 128 allows for direct user comparison of H₂S levels in the first assay sample 114 with a predetermined color of a spectral reference standard 120 and by the simple press of a button that acts as a user interface 144, an excitation source 152 excites the second assay sample 116 and the second spectral reference standard 122 with UV excitation light while a window 154 with an optical filter 156 that filters out the UV excitation light while allowing the spectral response with a longer wavelength to pass the optical filter 156 for direct optical comparison with the fluorescent spectral response of the second spectral reference standard. In some examples, the optical filters 156 are 500 nm long pass filters that selectively transmit wavelengths above 500 nm while blocking shorter wavelengths. In certain example implementations, a cover is employed to prevent the user from looking directly at the excitation source 152.

Selecting the kit based on the assay reader available and suitable for a selected testing environment provides rapid and sensitive detection of both H₂S and SRB enzymatic activity. In usage scenarios requiring higher sensitivity and specificity, particularly in complex matrices or when dealing with low concentrations, the advanced spectrometry assay reader 108b may be particularly well-suited. This reader can concurrently analyze both absorbance and fluorescence, enabling the use of a colorigenic reagent like Evan's Blue for H₂S detection alongside a fluorogenic reagent such as 7-azido-4-methylchromen-2-one for enhanced H₂S sensitivity, combined with SRB fluorogenic substrates like dipeptidyl-7-Amino-4-methylcoumarin for enzyme activity detection. Such implementation may be suitable for a more comprehensive analysis, differentiating between biologically mediated H₂S production and abiotic sources.

In situations where sample volume is limited or when rapid, single-sample analysis is needed, the mini assay reader 108c with its combination assay sample becomes advantageous, utilizing reagents that can interact synergistically, such as fluorogenic compounds for both H₂S and SRB detection that can be excited by a single UV source.

The selections of reagents included in the kit 128 may also be based upon environmental factors such as temperature, pressure, and salinity, which can affect reagent performance and stability, especially in challenging oil and gas industry settings.

By providing a system 100, 300, 400 with several suitable assay reader configurations and a suitable colorigenic reagents, fluorogenic reagents, and other components that have been specifically designed and/or tested for rapid multimodal detection of anaerobic sulfate-reducing bacteria via integrative spectral comparison, test results, and mitigation recommendations can be optimized across a wide range of fluid test samples and environmental conditions.

FIG. 4 is a schematic block diagram illustrating a system 400 for field use in rapid multimodal detection of anaerobic SRB concurrent with detection of H₂S in a fluid test sample via integrative spectral comparison of corresponding spectral parameters 132, 138 resulting from a combination assay sample 118, according to one or more aspects of the present disclosure.

In various aspects, the system 400 is substantially similar to the systems 100 and 300 depicted respectively in FIGS. 1 and 3. The system 400 depicts a mini assay reader 108c with a first sample compartment 110 into which a combination assay sample 118 is inserted for integrative spectral comparison of H₂S and anaerobic SRB levels in a fluid test sample 104 to be performed concurrently based on spectral parameters resulting the introduction of a fluid test sample 104 into an assay container 112c containing a kit 128 that includes: (i) a set of one or more first reagents 130 formulated to indicate, via a first set of spectral parameters 132, a level of H₂S in the fluid test sample (ii) a set of one or more second reagents 136 formulated to indicate, via a first set of spectral parameters 132, a level of anaerobic SRB 102 in the fluid test sample, and (iii) one or more oxygen scavengers 140 for maintaining an anerobic environment within the combination assay sample 118 during analysis.

In certain examples, at least one of the one or more first reagents 130 and the one or more second reagents 136 are formulated to exhibit at least one spectral parameter of the first set of spectral parameters and/or the second set of spectral parameters in response to excitation by ultraviolet (UV) light via the excitation source 152. In some examples, at least one of the first set of spectral parameters and the second set of spectral parameters includes a wavelength and/or wavelength range of peak absorbance in response to exposure to the UV light. In some examples, the first set of spectral parameters includes detection of emitted visible light in response to exposure to excitation by ultraviolet (UV) light.

In some examples, the detection of H₂S and bacterial activity occurs via the same one or more assay containers 112. In some examples, both the one or more first reagents 130 and the one or more second reagents 136 are colorogenic reagents. In some examples, both of the one or more first reagents 130 and the one or more second reagents 136 are fluorogenic reagents. In some examples, the one or more first reagents 130 are formulated to react with the H₂S in the fluid test sample 104.

In certain implementations, a combination assay sample 118 as depicted in FIG. 4 includes an innovative combination assay sample 118 for the concurrent detection of H₂S and SRB enzymatic activity in a fluid test sample by combining one or more first reagents 130, one or more second reagents 136, and one or more oxygen scavenger 140 in the same combination assay container 112c so that upon introduction of the fluid test sample 104 into the combination assay container 112c, a combination assay sample 118 is formed.

In certain implementation, the assay utilizes a synergistic cascade fluorescence mechanism, employing two distinct fluorogenic reagents to achieve enhanced sensitivity and selectivity. The first reagent, 7-azido-4-methylchromen-2-one, serves as an H₂S probe, undergoing a reduction of its aromatic azide moiety in the presence of tetrasodium; 4-amino-6-[[4-[4-[(8-amino-1-hydroxy-5,7-disulfonatonaphthalen-2-yl)diazenyl]-3-methylphenyl]-2-methylphenyl]diazenyl]-5-hydroxynaphthalene-1,3-disulfonate to yield fluorescent 7-azido-4-methylchromen-2-one. This initial reaction exhibits an emission peak at approximately 450 nm when excited by UV light.

The second reagent, (6′-acetyloxy-3-oxospiro[2-benzofuran-1,9′-xanthene]-3′-yl) acetate, functions as an indicator of SRB enzymatic activity. Upon hydrolysis by esterases present in metabolically active SRB cells, (6′-acetyloxy-3-oxospiro[2-benzofuran-1,9′-xanthene]-3′-yl) acetate releases 3′,6′-dihydroxyspiro[2-benzofuran-3,9′-xanthene]-1-one, a highly fluorescent compound with an emission peak above 500 nm. One innovative aspect of this combination assay lies in its cascading mechanism, where the fluorescence emitted by 7-azido-4-methylchromen-2-one, (resulting from H₂S detection) serves as an excitation source for the 3′,6′-dihydroxyspiro[2-benzofuran-3,9′-xanthene]-1-one produced by SRB activity. This cascading effect yields an overall spectral response enabling concurrent detection of both H₂S levels and SRB metabolic activity by detecting the spectral emission response of the 3′,6′-dihydroxyspiro[2-benzofuran-3,9′-xanthene]-1-one indicating with one spectral response that both H₂S and SRB enzymatic activity are detected in the assay sample.

The assay is conducted in a suitable buffer system, such as phosphate or MES (2-(N-morpholino) ethanesulfonic acid) buffer, optimized to maintain stable pH conditions and compatibility with both H₂S detection and SRB enzymatic activity measurements.

A cascading fluorescence combination assay can be implemented with a single assay container and a single spectral reference standard for comparison with only the fluorogenic spectral response of the anaerobic SRB enzymatic detection regent because only when a predetermined level of H₂S is present in the fluid test sample will there be a fluorogenic spectral emission that serves as an excitation source for a second fluorogenic spectral response when a fluorescence reporter molecule is cleaved by anaerobic SRB enzymatic activity. Accordingly, the disclosed method provides a powerful tool for monitoring and managing microbial-induced corrosion and souring processes. By integrating the detection of H₂S and SRB activity, the assay offers improved sensitivity and specificity compared to traditional methods. The cascading fluorescence mechanism enables the detection of low concentrations of both H₂S and SRB enzymatic activity, making it particularly suitable for early detection and prevention of microbial-related issues in industrial settings.

In some examples, the system 100, 400, includes an advanced spectrometry sample reader 108a that enables a combination assay sample 118 to be formed upon mixture of a fluid test sample 104 with various combinations of one/or more colorogenic and/or fluorogenic first reagents 130 formulated to indicate levels of H₂S, combined with one or more complementary types (e.g., respectively fluorogenic and/or colorogenic second reagents 136 formulated to indicate levels of anaerobic SRB enzymatic activity with oxygen scavengers 140 formulated to maintain an anaerobic environment within the assay container 112b.

Significantly, in certain examples, as depicted in FIG. 4, the advanced spectrometry assay reader 108b is a dual-detection spectrometry instrument that utilizes a unique configuration of excitation sources 152 and two detectors 162 to facilitate the measurement of absorbance, fluorescence, and scattered light from a fluid sample. The excitation sources, such as laser diodes, are positioned to direct their beams through the fluid test sample, with their arrangement optimized to provide high-intensity and stable illumination. In some examples the excitation sources 152 are modulated at a fixed frequency to enhance the signal-to-noise ratio, allowing for improved isolation and detection of signals associated with scattered or fluorescent light emissions. In certain examples, a first detector 162a is located on the opposite side of the fluid test sample, in alignment with the illumination angle or path of the excitation sources and is used to measure the absorbance or transmission of light that passes directly through the sample. This configuration enables precise quantification of light attenuation caused by the analytes in the solution.

A second detector 162b is positioned at an offset angle 164 relative to the illumination angle or path of the excitation sources, such that it is generally orthogonal to the direct transmission path or within a suitable range of angles optimized for detecting scattered or fluorescent light. By placing the second detector 162 at the offset angle 164, typically 90 degrees or within an predetermined range of this value, interference from transmitted light is minimized, thereby enhancing the sensitivity to light emitted by fluorescence responses or scattered by the analytes. The angular placement of the second detector 162b can be adjusted depending on the requirements of the specific measurement, allowing flexibility to optimize the detection of fluorescence or scattered light signals.

This arrangement of excitation sources 152 and detectors 162a, 162f within the depicted advanced spectrometry assay reader 108b enables dual-mode spectrometry, combining absorbance measurements with the capability to detect fluorescence and scattering. The instrument's configuration, which includes angularly offset detection for fluorescence or scattering alongside direct transmission detection, makes it suitable for multi-mode analysis of analytes in liquid samples. This design is particularly advantageous for integrative spectral comparison of a combination assay sample for determining levels of H₂S and anaerobic SRB enzymatic concurrently within a single assay container using a kit that includes one or more oxygen scavengers and/or oxygen scavenging enhancers.

Included among the innovative and advantages technical effects of this approach are the following. By predosing the sample container with the one or more first reagents, the one or more second reagents, and the one or more oxygen scavengers, a closed anaerobic environment is maintained for perform integrative spectral comparison analysis of H₂S levels and anaerobic SRB enzymatic activity in the same assay sample and that singular measurement environment is maintained measurements be analyzed over selected periods of time.

The capability of such an advanced spectrometry assay sample reader 108b to perform integrative spectral comparison of spectral responses of fluorescence and absorbance enables a user to determine for both the one or more first reagents formulated to indicate via a first set of spectral parameters a level of hydrogen sulfide (H₂S) in a fluid test sample as well as for the one or more second reagents formulated to indicate via a second set of spectral parameters, a level of enzymatic activity of one or more species of anaerobic sulfate-reducing bacteria (SRB) in the fluid test sample whether to use colorigenic or fluorogenic reagents. The reagents listed in Table 1, Table 2, Table 3, and Table 4 are advantageous but non-limiting examples of suitable reagents that can be combined appropriately for a particular SRB detection circumstance by a skilled artisan who has the guidance of this disclosure based on various factors such as type of fluid sample, species of anaerobic SRB to test for, fluid test sample extraction environment, desired assay analysis time, cost, and information needed to provide appropriate mitigation recommendations.

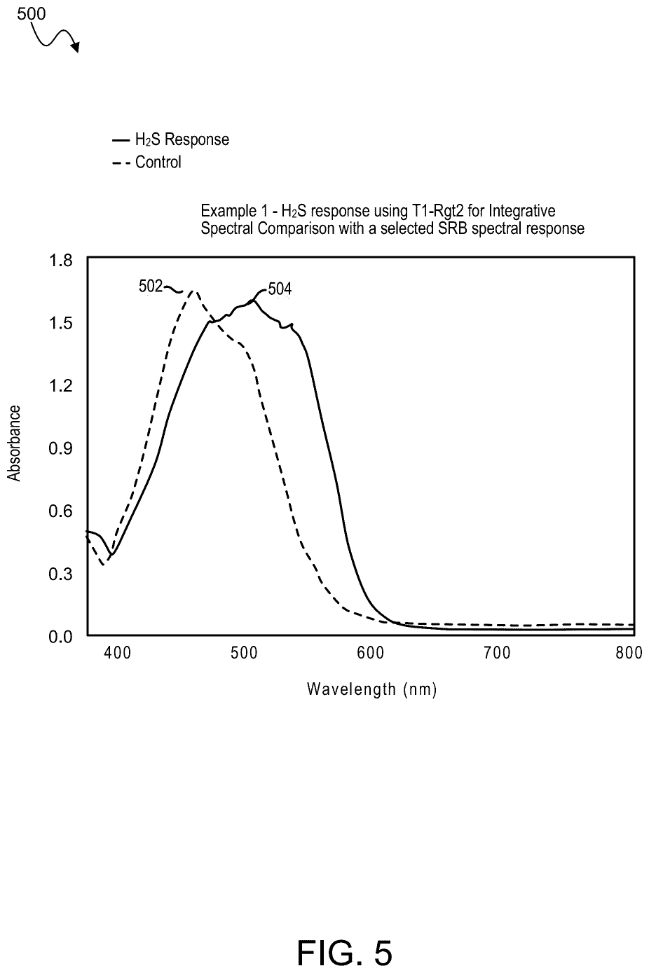

In the working examples shown in FIGS. 5, 6, 7, and 8, there are at least two types of spectral parameter comparisons that can be made. One type of comparison is a comparison of the spectral response of an assay sample formed by contacting one or more selected reagents with a fluid test with a spectral response of a spectral reference standard for indicating relative levels of the measured response of the assay sample with the measured response of the spectral reference standard.

However, in contrast to existing measurement approaches that measure H₂S levels or SRB levels but fail to do so in an integrative way, in the working examples shown in FIGS. 5, 6, 7, and 8, an annotation above the graph of selected spectral parameters (for example absorbance over a range of wavelengths) serves to highlight that fact that these graphs serve merely to provide a detailed illustration the spectral response of a first target analyte of the integrative spectral comparison e.g., (in FIGS. 5, 6, 7, and 8 this was H₂S) and that significant improvements over existing approaches is achieved by the technologies disclosed herein because they enable integrative spectral comparison to be rapidly performed for the same fluid test sample concurrent within 20-30 minute by comparing the spectral response of the first target analyte with a spectral response of a second target analyte e.g., anaerobic SRB enzymatic activity. By contrast, as described above with respect to FIG. 2, existing technologies often take days or even weeks or sample extraction, serial titration, incubation, and other steps to be performed to determine SRB levels. Because such existing approaches do not concurrently integrate or compare the spectral responses of both H₂S and anaerobic SRB, their accuracy, effectiveness, and general usefulness is limited.

FIG. 5 is a graph 500 illustrating detection of H₂S via a first reagent 130 that is colorigenic, according to one or more aspects of the present disclosure. As illustrated in the graph 500, in some examples, the first reagent 130 includes p-[(E)-4-Hydroxy-1-naphthylazo]benzenesulfonic acid. As shown by the graph 500, examples of the present disclosure include first reagents 130 formulated to indicate the presence of H₂S in a fluid test sample 104 via spectral parameter(s) that include a wavelength range of peak absorbance. Spectral response 502 illustrates absorbance as a function of wavelength (nm) for a fluid test sample 104 and/or spectral reference standard 120, 122, 124 having no H₂S or very little H₂S. Spectral response 504 illustrates the same for a fluid test sample 104 and/or spectral reference standard 120, 122, 124 having approximately 10 mg/L of H₂S. As shown in the graph 500, the peak absorbance for a fluid test sample 104 including H₂S is over 500 nm, whereas the peak absorbance for a fluid test sample 104 with no H₂S is less than 500 nm. In some examples, detection of absorbance is accomplished visually, via colorimetry, and/or using a spectrometer.

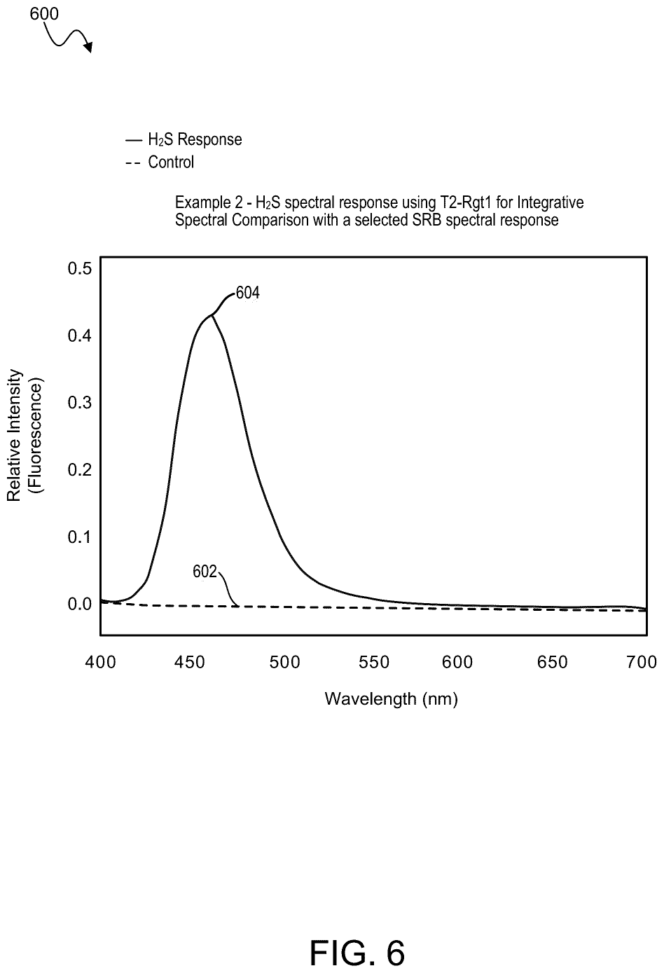

FIG. 6 is a graph 600 illustrating detection of H₂S, according to one or more aspects of the present disclosure. The graph 600 illustrates the detection of H₂S arising from sulfate-reducing bacteria using 7-azido-4-methylchromen-2-one via fluorescence intensity in response to excitation with UV light. The graphed spectral response 602 is for a baseline response of 0 mg/L of H₂S, and the spectral response 604 is a positive indication of H₂S, specifically of 10 mg/L. As shown in FIG. 6, a fluid test sample 104 having 10 mg/L of H₂S has a peak relative intensity of fluorescence at a wavelength of about 450 nm, whereas the fluid test sample 104 and/or spectral reference standard 120, 122, 124 having no H₂S has a smaller peak relative intensity of fluorescence at a wavelength of less than 400 nm. As such, in some examples, a spectral reference standard 120, 122, 124 and/or fluid test sample 104 without H₂S fluoresces at a different wavelength than a fluid test sample 104 containing H₂S. In some examples, the first set of spectral parameters and/or second set of spectral parameters includes a wavelength range of peak absorbance.

FIG. 7 is a graph 700 illustrating detection of H₂S via 5-[(3-carboxy-4-nitrophenyl)disulfanyl]-2-nitrobenzoic acid, according to one or more aspects of the present disclosure. In the examples shown by the graph 700, the kit 128 includes a first reagent 130 including 5-[(3-carboxy-4-nitrophenyl)disulfanyl]-2-nitrobenzoic acid and formulated to detect H₂S.

A spectral response 704 (graphed as a solid line) was measured of an assay sample that was formed by contacting a first reagent 5-[(3-carboxy-4-nitrophenyl)disulfanyl]-2-nitrobenzoic acid (listed in Table 1 as T1-Rgt1) with a fluid test sample 104. The spectral response 704 indicates absorbance as a function of wavelength (nm) of light emitted by a fluid test sample 104 including at least 10 mg/L of H₂S. For direct comparison, a spectral response 702 (graphed as a dashed line) corresponding to another fluid test sample containing no or very little H₂S that served as a spectral reference standard 120, 122, 124.

In the examples illustrated in FIG. 7, detection of the H₂S can occur by viewing the fluid test sample 104 visually, via colorimetry, and/or using a spectrometer. Absorbance is a unitless measure of the amount of light of a particular wavelength that passes through a volume of liquid, relative to the maximum possible amount of light available at that wavelength. Absorbance readings are unitless because they are calculated from a ratio of the intensity of light transmitted through the sample (I) to the intensity of light transmitted through a blank (Io). Absorbance=log 10 (Io/I). Absorbance is quite often reported in “Absorbance Units” or AU. Accordingly, optical density is measured in ODU, which are equivalent to AU cm−1. The higher the optical density, the lower the transmittance.

As shown in FIG. 7, a fluid test sample 104 without H₂S for example, a spectral reference standard has a peak absorbance of less than 0.7 absorbance units (AU) at a wavelength of approximately 400 nm. The fluid test sample 104 containing H₂S, had an assay sample spectral response 704 with a peak absorbance at approximately the same wavelength as the peak absorbance of a spectral reference standard spectral response 702 (for example, around 400 nm). Notably, the absorbance for the assay sample spectral response 704 is higher, at above 1.0 and nearly 1.2. In the working example, the assay sample was formed when a first reagent 5-[(3-carboxy-4-nitrophenyl)disulfanyl]-2-nitrobenzoic acid was contacted with a fluid test sample containing H₂S and a yellow to orange color was observed.

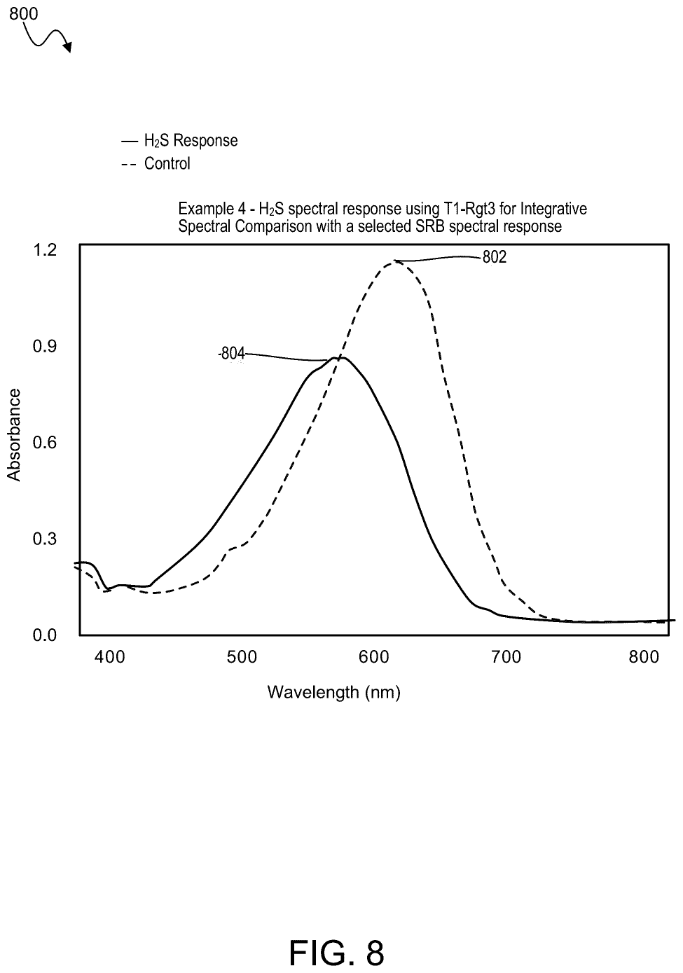

FIG. 8 is a graph 800 illustrating a working example of detection of H₂S via a colorigenic reagent, according to one or more aspects of the present disclosure. As shown in the graph 800, the first reagent 130 included a colorigenic reagent, 4-Amino-6-[(E)-(E)-1′b-(8-amino-1-hydroxy-5,7-disulfo-2-naphthylimino)-3,3′-dimethyl-1′a-benzidineylideneamino]-5-hydroxy-1,3-naphthalenedisulfonic acid (T1-Rgt3). The first set of spectral parameters and/or second set of spectral parameters include a wavelength of peak absorbance. The presence of H₂S was detected by determining the wavelength of the peak absorbance of the light emitted from the fluid test sample 84 when excited by light.

The spectral response 802 illustrates absorbance as a function of wavelength (nm) for a fluid test sample 84 and/or spectral reference standard 120, 122, 124 having no or very little H₂S. A spectral response 804 of assay sample form by contacting a first reagent T1-Rgt3 with a highly-contaminated fluid test sample with H₂S levels of approximately 50 mg/L. As shown in FIG. 8, the assay sample had a peak absorbance in the wavelength range of about 575 nm, whereas another fluid test sample with no H₂S serving as a spectral reference standard had a peak absorbance in the wavelength range of approximately 620 nm. Descriptively, in various examples, fluid test samples containing H₂S that have been exposed to 4-Amino-6-[(E)-(E)-1′b-(8-amino-1-hydroxy-5,7-disulfo-2-naphthylimino)-3,3′-dimethyl-1′a-benzidineylideneamino]-5-hydroxy-1,3-naphthalenedisulfonic acid change from a blue to a purple color.

FIG. 9 is a graph 900 illustrating detection of bacterial enzymes from SRB via a fluorescent reagent, according to one or more aspects of the present disclosure. Specifically, FIG. 9 illustrates detection of bacterial enzymes in the fluid test sample 104 using (6′-acetyloxy-3-oxospiro[2-benzofuran-1,9′-xanthene]-3′-yl) acetate as the second reagent 136. The spectral response 902 illustrates results from a fluid test sample having no sulfate-reducing bacteria to serve as a control sample. The spectral response 904 illustrates results from a fluid test sample 104 having sulfate-reducing bacteria. As shown in FIG. 9, the control sample having no sulfate-reducing bacteria shows little fluorescence when excited at 450 nm. On the other hand, the fluid test sample having sulfate-reducing bacteria, has a spectral response 904, that exhibits fluorescence above 500 nm when excited at 450 nm.

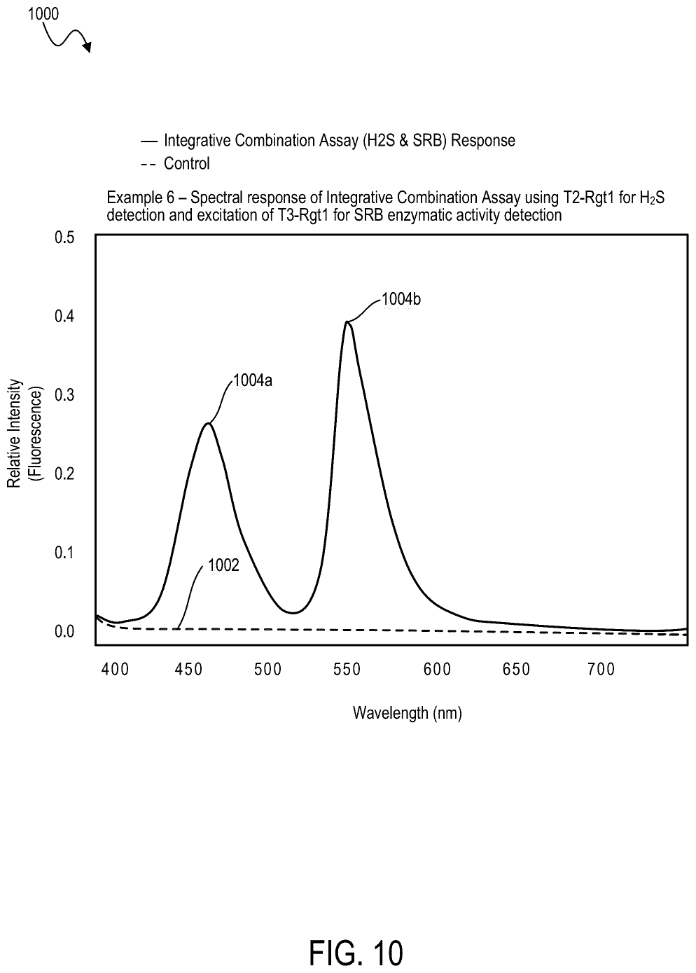

FIG. 10 is a graph 1000 of a working example illustrating simultaneous detection of H₂S and anaerobic SRB enzymatic activity in a fluid test sample 104, according to one or more aspects of the present disclosure. The graph 1000 illustrates results of an assay sample forms using a kit 128 including 7-azido-4-methylchromen-2-one as the first reagent 130 and (6′-acetyloxy-3-oxospiro[2-benzofuran-1,9′-xanthene]-3′-yl) acetate as the second reagent 136. To produce the response depicted in the graph 1000, the fluid test sample 104 was excited with UV light from an excitation source 152. The kit 128 used included a first reagent 130, a second reagent 136, and the fluid test sample 104. The spectral reference standard spectral response 1002 illustrates a peak relative intensity of fluorescence as a function of wavelength (nm) for a spectral reference standard containing no sulfate-reducing bacteria that served as a control sample.

The assay sample spectral response 1004a, 1004b shows two peaks of relative intensity of fluorescence for a fluid test sample 104 containing sulfate-reducing bacteria. For direct comparison the spectral reference standard spectral response 1002 with no SRB exhibits little or no fluorescence when excited with UV light, whereas the assay sample spectral response 1004a exhibited a relative intensity fluorescence peak response at a wavelength of about 450 nm from reaction of 7-azido-4-methylchromen-2-one with H₂S. The emission response from detecting H₂S was then absorbed by a reaction of (6′-acetyloxy-3-oxospiro[2-benzofuran-1,9′-xanthene]-3′-yl) acetate with enzymes from SRB in the fluid test sample which then in turn emitted a fluorescence response with a peak intensity wavelength of about 550 nm. This working example illustrates one implementation of a combination assay sample where the first reagents, second reagents, and oxygen scavenger are combined in a single assay sample container. Because the spectral response at 550 nm indicating levels of anaerobic SRB was enabled by the emission of excitation at 450 nm from a reaction of H₂S in the fluid test sample with the first reagent 7-azido-4-methylchromen-2-one, a tangible spectral reference standard 124 that emits fluorescence at a peak intensity wavelength of about 550 could be utilized, for example, for direct comparison using a mini assay reader 108c.

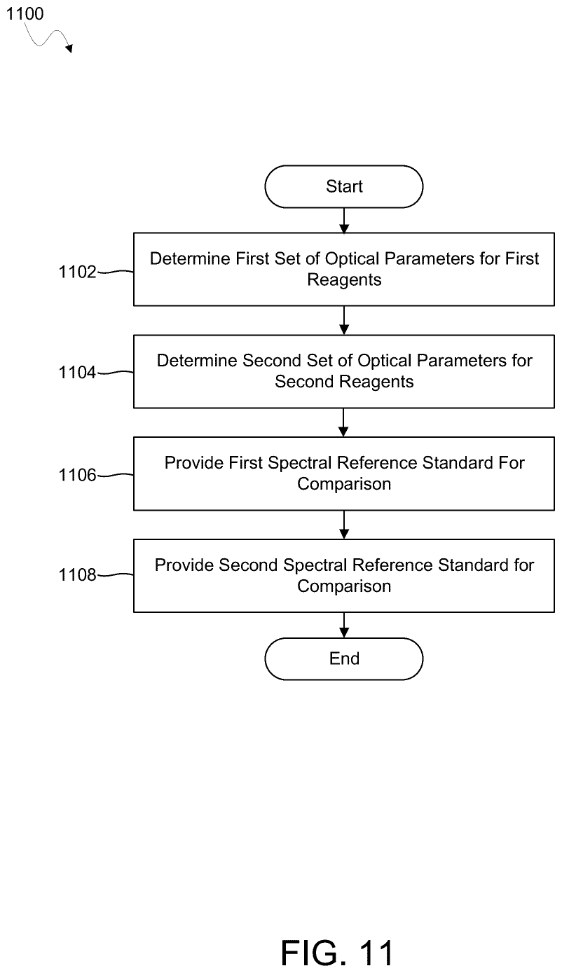

FIGS. 11-15 illustrate various aspects of methods for creating and/or providing various kits 128 for rapid multimodal detection of anaerobic sulfate-reducing bacteria via integrative spectral comparison. As evidenced by the overview 200 depicted in FIG. 2, existing approaches for assessing indications of SRB in fluid test samples include the serial dilution culturing approach 202, the ATP photometry approach 204, the filtered enzyme substrate dye approach 206, and molecular biological methods approach 208, the existence of chemical reagents and protocols to detect certain aspects of SRB in test fluid over decades has not led to a system, assay reader, or kit for rapid multimodal detection of anaerobic sulfate-reducing bacteria via integrative spectral comparison. The methods disclosed in FIGS. 11-15 highlight certain innovative steps of these methods that a manufacturer, vendor, or other provider can practice based on the guidance providing in the present disclosure to successfully provide a system, apparatus, or kit for rapid multimodal detection of anaerobic sulfate-reducing bacteria via integrative spectral comparison.

It should be noted as demonstrated by the rapidity, accuracy, and usability of the working examples and technologies disclosed herein, that the kits produced by these methods provide synergistic results achieved through the specific combination and sequence of kit components where the actionable results of the combined steps are significantly greater in terms of rapidity, accuracy, field use, and usability than for example, the sum of the individual results of prior art reagents used in prior art approaches.

FIG. 11 is a schematic flowchart diagram illustrating a method 1100 for detecting H₂S, according to one or more aspects of the present disclosure.

In some examples, the method 1100 includes determining 1102 a first set of spectral parameters for one or more first reagents 130 formulated to indicate via, the spectral parameters, one or more predetermined levels of hydrogen sulfide (H₂S) in a fluid test sample 104.

In some examples, the method 1100 also includes determining 1104 a second set of spectral parameters for one or more second reagents formulated to indicate, via the spectral parameters, one or more predetermined levels of enzymatic activity of one or more species of anaerobic sulfate-reducing bacteria in the fluid test sample 104.

In some examples, the method 1100 includes providing 1106 a first spectral reference standard for comparison with one or more first spectral parameters indicative of one or more predetermined levels of hydrogen sulfide (H₂S) in a fluid test sample by interaction with the one or more first reagents. In certain examples, the first spectral reference standard includes a set of spectral parameters representative of one or more predetermined levels of hydrogen sulfide (H₂S) in a first spectral reference configured for performing a first comparison with a first spectral signal to be generated in response to interaction of the one or more first reagents with H₂S upon contact with the fluid test sample 104.

In some examples, the method 1100 includes providing 1108 a second predetermined spectral reference standard for comparison with one or more second reaction results. In some examples, the second predetermined calibration standard includes a set of spectral parameters representative of or more predetermined levels of enzymatic activity of one or more species of anaerobic sulfate-reducing bacteria in a second spectral reference based on a second comparison with a second spectral signal. In some examples, the second spectral signal is generated in response to microbial interaction with a combination of the one or more second reagents 136 and one or more oxygen scavengers 140. In some examples, the one or more oxygen scavengers 140 are formulated to, upon contact with the fluid test sample 104, maintain an anaerobic environment for the one or more species of anaerobic sulfate-reducing organisms.

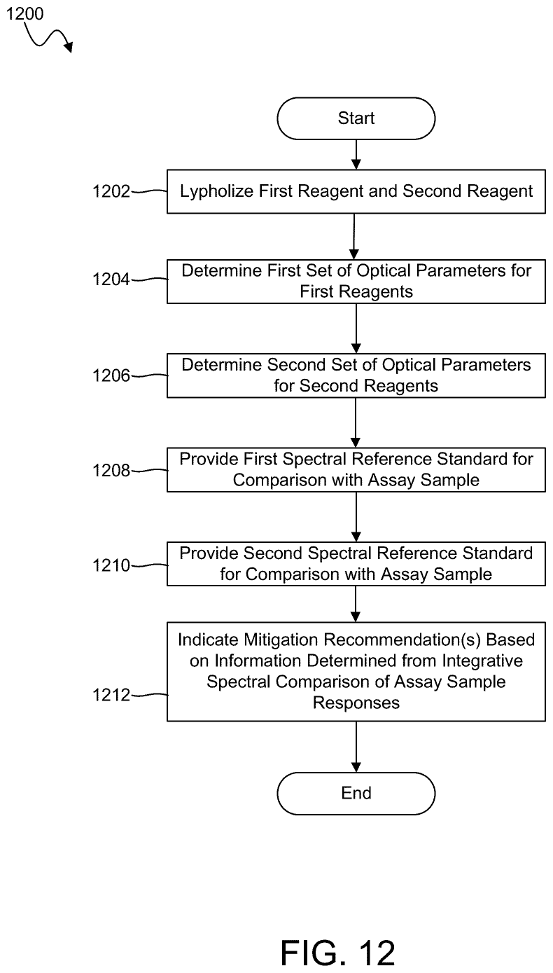

FIG. 12 is a schematic flowchart diagram illustrating a method 1200 for detecting H₂S via one or more lyophilized reagents, according to one or more aspects of the present disclosure. In some examples, the method 1200 includes lyophilizing 1202 the first reagent 130 and the second reagent 136.

In various examples, the method 1200 includes determining 1204 a first set of spectral parameters for one or more first reagents 130 formulated to indicate via, the spectral parameters, one or more predetermined levels of hydrogen sulfide (H₂S) in a fluid test sample 104.

In some examples, the method 1200 also includes determining 1206 a second set of spectral parameters for one or more second reagents 136 formulated to indicate, via the spectral parameters, one or more predetermined levels of enzymatic activity of one or more species of anaerobic sulfate-reducing bacteria in the fluid test sample 104.

In certain examples, the method 1200 includes providing 1208 a first spectral reference standard. In some examples, the first spectral reference standard includes a set of spectral parameters representative of one or more predetermined levels of hydrogen sulfide (H₂S) in a first spectral reference configured for performing a first comparison with a first spectral signal to be generated in response to a reaction of the one or more first reagents with H₂S upon contact with the fluid test sample 104.

In one or more examples, the method 1200 includes providing 1210 a second predetermined spectral reference standard. In some examples, the second predetermined calibration standard includes a set of spectral parameters representative of or more predetermined levels of enzymatic activity of one or more species of anaerobic sulfate-reducing bacteria in a second spectral reference based on a second comparison with a second spectral signal. In some examples, the second spectral signal is generated in response to microbial interaction with a combination of the one or more second reagents 136 and one or more oxygen scavengers 140. In some examples, the one or more oxygen scavengers 140 are formulated to, upon contact with the fluid test sample 104, maintain an anaerobic environment for the one or more species of anaerobic sulfate-reducing organisms.

In certain examples, the method 1200 includes indicating 1212 one or more mitigation recommendations to reduce levels of anaerobic sulfate-reducing bacteria in a source of fluid for the fluid test sample based on a combination of information determined from the first comparison and the second comparison.

As described and depicted in various of the foregoing examples, the presence of sulfate-reducing bacteria (SRB) with the concomitant presence of dissolved hydrogen sulfide (H₂S) gas can be mitigated through several alternatives. Treatment includes the use of biocides including chlorine-based options (chlorine gas, hypochlorite, chlorine dioxide and the like) as well as similar bromine-based adducts, 2,2-dibromo-3-nitrilopropionamide (DBNPA), thiocayanate or isothiazolinones. Many of these options chemically degrade dissolved H₂S and can also inhibit and destroy SRB. Glutaraldehyde-based biocides can be quite effective against encapsulated SRB.

In certain circumstances, it is beneficial to conduct the microbial test and the hydrogen sulfate test in separate reaction vessels. For example, when the microbial test is negative and the presence of H₂S is indicated, it may be appropriate to treat for dissolved H₂S only. This circumstance can be due to the presence of residual SRB metabolic H₂S production or natural geo-mineral formation. Specific treatment for H₂S includes aeration, metal oxide-based adsorbents, sodium chlorite or triazine.

As a further example of the advantages of the methods disclosed herein, when microbial activity is present but little or no H₂S is detected, it can indicate that the SRB are in a metabolic lag-stage and not producing detectable amounts of H₂S or that H₂S levels have decreased through other physical or chemical processes during storage or transportation. Treatment using biocides suitable for SRB in a metabolic lag-stage can thus be indicated. Some biocides including oxidizing biocides such as chlorine (Cl2) or hypochlorous acid (HOCl) may be suitable in certain circumstances where the method indicates little SRB enzymatic activity but are less effective at higher pH levels (above 8.0), which may limit their efficacy in environments where SRB are active.

Various nitrate and nitrite compounds can inhibit the metabolic processes of SRB, reducing H₂S production and in many industrial settings is effective in controlling SRB activity. Inducing metabolic stress in SRB using nitrite in combination with glutaraldehyde can enhance the kill rate of biocides. This approach leverages the stress response of bacteria to make them more susceptible to biocidal action, which can be particularly useful during the lag phase.

Mitigation recommendations, in certain cases, may include use green biocide enhancers improve the penetration and effectiveness of biocides, making them more suitable for treating SRB in various stages of their metabolic cycle, including the lag phase. For examples, one such green biocide enhancer is ethylenediaminedisuccinate or EDDS, a biodegradable chelator that enhances the efficacy of glutaraldehyde in treating SRB biofilms. EDDS reduces the required dosage of glutaraldehyde, making it a more environmentally friendly option for industrial applications, particularly in the oil and gas industry. The system, kit, and methods disclosed herein advantageously indicate a level of SRB enzymatic activity which is an indication of SRB metabolic activity. This information is useful because, during a lag phase, bacteria are preparing for growth but not actively dividing, which can make them less susceptible to biocides that target cell division. According, in such cases, the method may include a mitigation recommendation to utilize EDDS to improve the penetration of biocides into the biofilm matrix during the lag phase.

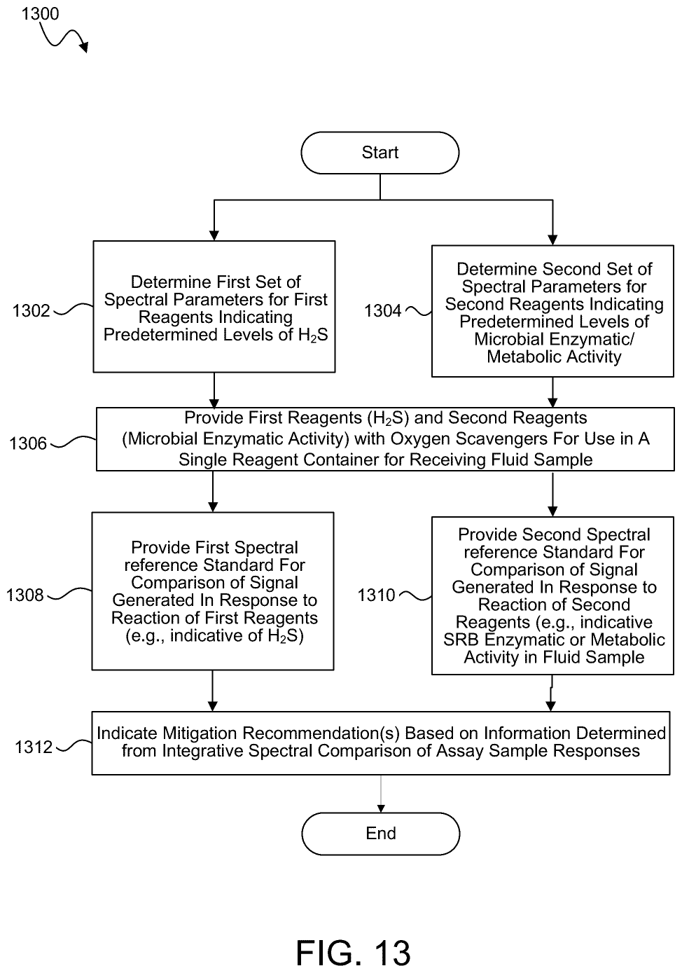

FIG. 13 is a schematic flowchart diagram illustrating a method 1300 for detecting H₂S via one or more assay containers, according to one or more aspects of the present disclosure.

In various examples, the method 1300 includes receiving 1302 a first reagent 130 and a second reagent 136 in a container 112 concurrently. In some examples, the determining 1304 a first set of spectral parameters for one or more first reagents 130 formulated to indicate via, the spectral parameters, one or more predetermined levels of hydrogen sulfide (H₂S) in a fluid test sample 104.

In some examples, the method 1300 also includes determining 1306 a second set of spectral parameters for one or more second reagents 136 formulated to indicate, via the spectral parameters, one or more predetermined levels of enzymatic activity of one or more species of anaerobic sulfate-reducing bacteria in the fluid test sample 104.

In one or more examples, the method 1300 includes providing 1308 a first spectral reference standard. In some examples, the first spectral reference standard includes a set of spectral parameters representative of one or more predetermined levels of hydrogen sulfide (H₂S) in a first spectral reference configured for performing a first comparison with a first spectral signal to be generated in response to a reaction of the one or more first reagents with H₂S upon contact with the fluid test sample 104.

In certain examples, the method 1300 includes providing 1310 a second predetermined spectral reference standard. In some examples, the second predetermined calibration standard includes a set of spectral parameters representative of or more predetermined levels of enzymatic activity of one or more species of anaerobic sulfate-reducing bacteria in a second spectral reference based on a second comparison with a second spectral signal. In some examples, the second spectral signal is generated in response to microbial interaction with a combination of the one or more second reagents 136 and one or more oxygen scavengers 140. In various examples, the one or more oxygen scavengers 140 are formulated to, upon contact with the fluid test sample 104, maintain an anaerobic environment for the one or more species of anaerobic sulfate-reducing organisms.

In some examples, the method 1300 includes indicating 1312 one or more mitigation recommendations to reduce levels of anaerobic sulfate-reducing bacteria in a source of fluid for the fluid test sample based on a combination of information determined from the first comparison and the second comparison.

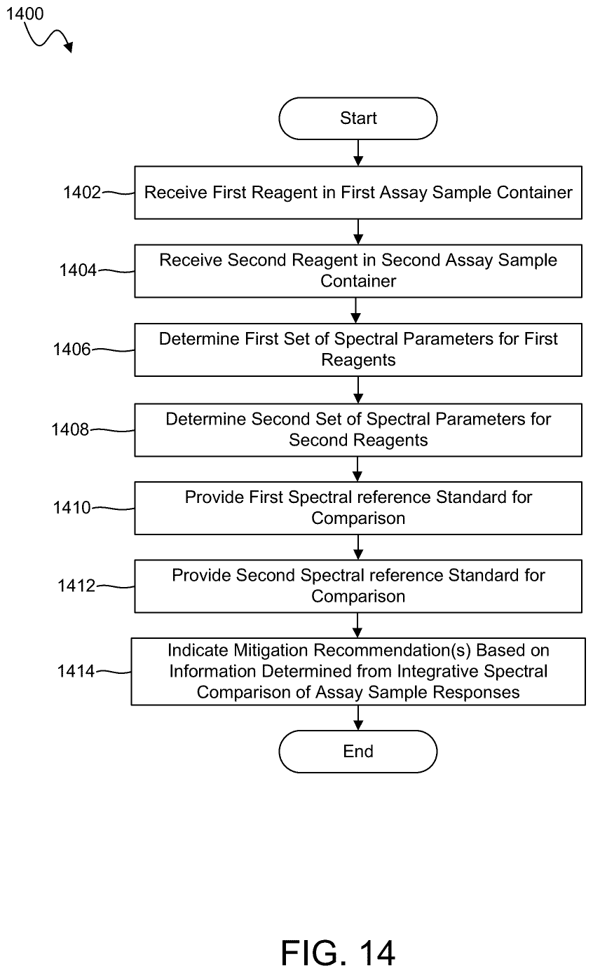

FIG. 14 is a schematic flowchart diagram illustrating a method 1400 for detecting H₂S via two or more assay containers 112, according to one or more aspects of the present disclosure. The method 1400 includes receiving 1402 a first reagent 130 in a first assay container 112a. In various examples, the method 1400 includes receiving 1404 a second reagent 136 in a second assay container 112b.

In certain examples, the method 1400 includes determining 1406 a first set of spectral parameters for the first reagents 130. The first reagents are formulated to indicate, via the spectral parameters, one or more predetermined levels of hydrogen sulfide (H₂S) in a fluid test sample 104. In some examples, the method 1400 includes determining 1408 a second set of spectral parameters for the second reagents 136. The second reagents 136 are formulated to indicate, via the spectral parameters, one or more predetermined levels of enzymatic activity of one or more species of anaerobic sulfate-reducing bacteria in the fluid test sample 104. In various examples, the spectral parameters are determined before first reagents 130 and/or second reagents 136 are received by the assay containers 112.

In certain examples, the method 1400 includes providing 1410 a first spectral reference standard comprising a set of spectral parameters representative of one or more predetermined levels of hydrogen sulfide (H₂S) in a first spectral reference configured for performing a first comparison with a first spectral signal to be generated in response a reaction of the one or more first reagents with H₂S upon contact with the fluid test sample 104.

In various examples, the method 1400 includes providing 1412 a second spectral reference standard comprising a set of spectral parameters representative of or more predetermined levels of enzymatic activity of one or more species of anaerobic sulfate-reducing bacteria in a second spectral reference based on a second comparison with a second spectral signal generated in response to microbial interaction with a combination of the one or more second reagents 136 and one or more oxygen scavengers 140 that are formulated to, upon contact with the fluid test sample 104, maintain an anaerobic environment for the one or more species of anaerobic sulfate-reducing organisms.

In some examples, the method 1400 includes indicating 1414 a mitigation recommendation based on a combination of information determined from the first comparison and the second comparison.

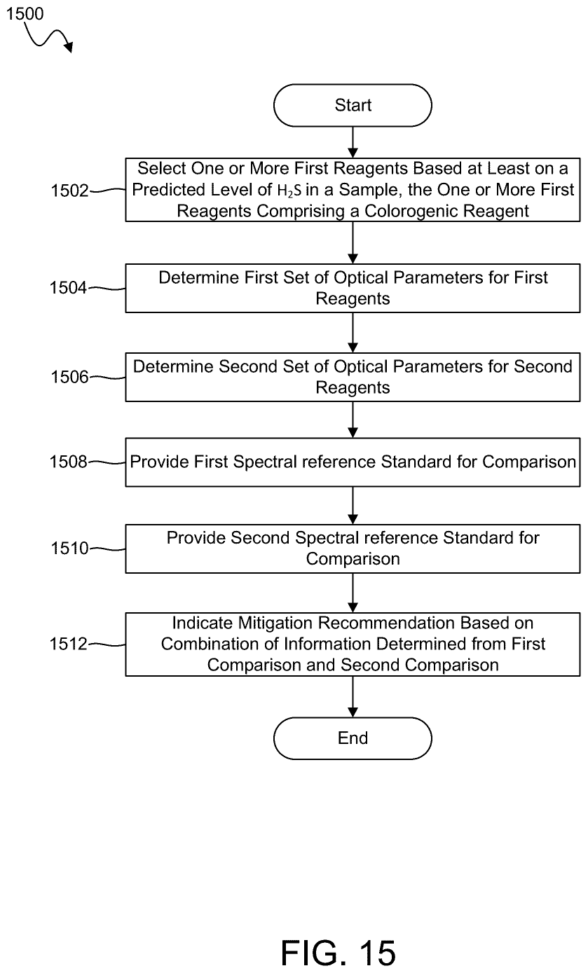

FIG. 15 is a schematic flowchart diagram illustrating a method 1500 for detecting H₂S via one or more reagents selected based at least in part on a predicted H₂S level, according to one or more aspects of the present disclosure. In some examples, the method 1500 includes selecting 1502 one or more first reagents 130 based at least in part on predicted levels of H₂S in a fluid test sample 104. The one or more first reagents 130 include a colorogenic reagent. In some examples, the method 1500 includes determining 1504 a first set of optical parameters for the first reagents 130. In some examples, the method 1500 includes determining 1506 a second set of optical parameters for the second reagents 136.

In some examples, the method 1500 includes providing 1508 a first spectral reference standard comprising a set of spectral parameters representative of one or more predetermined levels of hydrogen sulfide (H₂S) in a first spectral reference configured for performing a first comparison with a first spectral signal to be generated in response a reaction of the one or more first reagents with H₂S upon contact with the fluid test sample 104. In some examples, the method 1500 includes providing 1510 a second spectral reference standard comprising a set of spectral parameters representative of or more predetermined levels of enzymatic activity of one or more species of anaerobic sulfate-reducing bacteria in a second spectral reference based on a second comparison with a second spectral signal generated in response to microbial interaction with a combination of the one or more second reagents 136 and one or more oxygen scavengers 140 that are formulated to, upon contact with the fluid test sample 104, maintain an anaerobic environment for the one or more species of anaerobic sulfate-reducing organisms.

In various examples, the method 1500 includes indicating 1512 a mitigation recommendation based on a combination of information determined from the first comparison and the second comparison.

The examples provided in this disclosure are intended to illustrate various implementations and should not be construed as limiting the scope of the invention. The scope of the invention is defined solely by the claims that follow, and any modifications or variations that fall within the meaning and scope of those claims, including equivalents, are considered to be encompassed by the claims.

What is claimed is:

1. A system for field use in rapid multimodal detection of anaerobic sulfate-reducing bacteria (SRB) in a fluid test sample via integrative spectral comparison, the system comprising:
an assay reader comprising:
one or more sample compartments configured to receive:
one or more assay samples for comparison with respective spectral reference standards; and
one or more excitation sources configured to emit light at wavelength selected to facilitate colorigenic and/or fluorogenic analysis of the one or more assay samples; and
a kit for performing an integrative assay of anaerobic SRB-mediated components of the fluid test sample comprising:
one or more first reagents, the one or more first reagents formulated to form a first assay sample upon contact with a first portion of the fluid test sample and to indicate, via a first set of spectral parameters, a level of hydrogen sulfide (H₂S) in the fluid test sample;
one or more second reagents, the one or more second reagents formulated form a second assay sample upon mixing with a second portion of the fluid test sample and to indicate, via a second set of spectral parameters, a level of enzymatic activity of one or more species of anaerobic SRB in the fluid test sample; and
one or more oxygen scavengers combined with the one or more second reagents and formulated to, upon contact with the fluid test sample, maintain an anaerobic environment for the one or more species of anaerobic SRB.

2. The system of claim 1, wherein the assay reader comprises one or more windows through which one or more spectral parameters of the first set of spectral parameters and/or the second set of spectral parameters pertaining to the first assay sample and/or the second assay sample are determinable.

3. The system of claim 2, wherein:
the one or more first reagents are formulated to react with H₂S to yield a first fluorescent compound; and
the one or more second reagents are formulated to yield a second fluorescent compound when exposed to one or more species of anaerobic sulfate-reducing bacteria in the fluid test sample.

4. The system of claim 3, wherein the first set of spectral parameters and the second set of spectral parameters comprise visible fluorescence emitted from the fluid test sample by the first fluorescent compound and the second fluorescent compound in response to excitation by an ultraviolet (UV) light source.

5. The system of claim 4, wherein an integrative assay sample is formed upon contact with the fluid test sample into an individual assay container which contains the one or more first reagents for detecting H₂S and the one or more second reagents for detecting enzymatic activity of SRB.

6. The system of claim 5, wherein the assay reader comprises a spectrometry instrument configured to detect and analyze the first set of spectral parameters and the second set of spectral parameters pertaining of the integrative assay sample concurrently.

7. The system of claim 2, wherein at least one of the one or more windows comprises a filter that selectively filters light corresponding to excitation source wavelengths and passes light corresponding to excitation response wavelengths.

8. The system of claim 7, wherein the filter selectively filters UV excitation and passes visible light corresponding to excitation response wavelengths.

9. A kit, comprising:
one or more first reagents formulated to indicate via a first set of spectral parameters a level of hydrogen sulfide (H₂S) in a fluid test sample;
one or more second reagents formulated to indicate via a second set of spectral parameters, a level of enzymatic activity of one or more species of anaerobic sulfate-reducing bacteria (SRB) in the fluid test sample; and
one or more oxygen scavengers combined with the one or more second reagents and formulated to, upon contact with the fluid test sample, provide an anaerobic environment for the one or more species of anaerobic sulfate-reducing bacteria.

10. The kit of claim 9, wherein one or more of the one or more first reagents are formulated to produce the first set of spectral parameters having a longer wavelength range than an excitation source wavelength range used in connection with the one or more second reagents.

11. The kit of claim 9, wherein at least one spectral parameter of the first set of spectral parameters and the second set of spectral parameters includes a wavelength of light emitted in response to excitation by an ultraviolet (UV) light source.

12. The kit of claim 9, wherein the one or more oxygen scavengers comprise sodium dithionite.

13. The kit of claim 12, further comprising (2S)-2-amino-5-[[(2R)-1-(carboxymethylamino)-1-oxo-3-sulfanylpropan-2-yl]amino]-5-oxopentanoic acid that in a combination with sodium dithionite, synergistically enhances oxygen-scavenging properties of the combination.

14. The kit of claim 9, wherein the one or more second reagents comprise (6′-acetyloxy-3-oxospiro[2-benzofuran-1,9′-xanthene]-3′-yl) acetate and the first set of spectral parameters comprises emitted visible light in response to excitation by a UV light source.

15. The kit of claim 9, wherein the one or more second reagents formulated to indicate the level of enzymatic activity of one or more species of anaerobic SRB in the fluid test sample are selected from a group consisting of: (6′-acetyloxy-3-oxospiro[2-benzofuran-1,9′-xanthene]-3′-yl) acetate, 7-hydroxy-10-oxidophenoxazin-10-ium-3-one, Dipeptidyl-7-Amino-4-methylcoumarin, (4-nitrophenyl) phosphate; 5-Bromo-6-chloro-3-indolyl caprylate, and combinations thereof.

16. The kit of claim 9, wherein the one or more first reagents are formulated to indicate levels of H₂S in the fluid test sample by the first set of spectral parameters with a predetermined spectral wavelength range upon reaction with H₂S in the fluid test sample.

17. The kit of claim 9, wherein the one or more first reagents are formulated to include p-[(E)-4-Hydroxy-1-naphthylazo]benzenesulfonic acid for enhancing a spectral comparison of a reaction of H₂S with p-[(E)-4-Hydroxy-1-naphthylazo]benzenesulfonic acid, where the fluid test sample before contacting the one or more first reagents produced a yellow baseline color and/or a target wavelength to be detected is in a range of about 550 nanometers (nm) to about 590 nm.

18. The kit of claim 9, wherein the one or more first reagents are formulated to include tetrasodium and 4-amino-6-[[4-[4-[(8-amino-1-hydroxy-5,7-disulfonatonaphthalen-2-yl)diazenyl]-3-methylphenyl]-2-methylphenyl]diazenyl]-5-hydroxynaphthalene-1,3-disulfonate for enhancing detection of concentrated H₂S.

19. The kit of claim 9, wherein the one or more first reagents comprise 5-[(3-carboxy-4-nitrophenyl)disulfanyl]-2-nitrobenzoic acid, and the kit further comprises one or more buffering agents.

20. The kit of claim 9, wherein the one or more first reagents comprise 7-azido-4-methylchromen-2-one or 7-Nitro-4-(7-nitro-2,1,3-benzofurazan-4-ylthio)-2,1,3-benzofurazan.

21. The kit of claim 9, wherein the one or more first reagents and/or the one or more second reagents are lyophilized until contact with the fluid test sample.

22. The kit of claim 9, further comprising a spectral reference standard, the spectral reference standard provided to indicate, via at least one of the first set of spectral parameters and the second set of spectral parameters corresponding respectively to wavelengths indicative of reaction of the one or more first reagents with at least one of a baseline level of H₂S and to wavelengths indicative of reaction of the one or more second reagents with a baseline level of enzymatic activity of the one or more species of anaerobic sulfate-reducing bacteria.

23. A method for providing a kit for field use in rapid multimodal detection of anaerobic sulfate-reducing bacteria (SRB) in a fluid test sample via integrative spectral comparison, the method comprising:
determining a first set of spectral parameters for one or more first reagents formulated to indicate, via the first set of spectral parameters, one or more predetermined levels of hydrogen sulfide (H₂S) in a fluid test sample;
determining a second set of spectral parameters for one or more second reagents formulated to indicate, via the second set of spectral parameters, one or more predetermined levels of enzymatic activity of one or more species of anaerobic sulfate-reducing bacteria in the fluid test sample;
providing a first spectral reference standard comprising a set of spectral parameters representative of one or more predetermined levels of hydrogen sulfide (H₂S) in a first spectral reference configured for performing a first comparison with a first spectral signal to be generated in response a reaction of the one or more first reagents with H₂S upon contact with the fluid test sample; and
providing a second spectral reference standard comprising a set of spectral parameters representative of or more predetermined levels of enzymatic activity of one or more species of anaerobic sulfate-reducing bacteria in a second spectral reference based on a second comparison with a second spectral signal generated in response to microbial interaction with a combination of the one or more second reagents and one or more oxygen scavengers that are formulated to, upon contact with the fluid test sample, maintain an anaerobic environment for the one or more species of anaerobic sulfate-reducing organisms.

24. The method of claim 23, further comprising indicating one or more mitigation recommendations to reduce levels of anaerobic sulfate-reducing bacteria in a source of fluid for the fluid test sample based on a combination of information determined from the first comparison and the second comparison.
